(19) United States
(12) Patent Application Publication
(10) Pub. No.: US 2003/0194725 A1
(43) Pub. Date: Oct. 16, 2003

(54) Methods for identifying and validating potential drug targets

(76) Inventors: Tsvika Greener, Ness-Ziona (IL); Avishai Levy, Rishon--Le-Zion (IL); Yuval Reiss, Kiriat-Ono (IL); Danny Ben-Avraham, Tel-Aviv (IL); Iris Alroy, Ness-Ziona (IL)

Correspondence Address: ROPES & GRAY LLP, ONE INTERNATIONAL PLACE, BOSTON, MA, 02110-2624, US

(21) Appl. No.: 10/299,991

(22) Filed: Nov. 19, 2002

Related U.S. Application Data
(60) Provisional application No. 60/331,701, filed on Nov. 19, 2001.

(51) Int. Cl. C12Q 1/68; G01N 33/53; G06F 19/00; G01N 33/48; G01N 33/50

(52) U.S. Cl. 435/6; 435/7.1; 702/19; 702/20

(57) ABSTRACT

This application provides methods for identifying and validating potential drug targets. In one aspect, the application provides a systematic method of creating a database of related protein or nucleic acid sequences with annotations of the potential disease associations of the sequences; and a method for testing the potential disease associations by means of a biological assay and validating the disease association by either decreasing expression of the sequence of interest or increasing expression of the sequence of interest.

### RELATED APPLICATIONS

This application claims the benefit of the filing date of U.S. Provisional Application No. 60/331,701, filed Nov. 19, 2001, the specification of which is hereby incorporated by reference in its entirety.

### BACKGROUND OF THE INVENTION

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The task of deciphering which of these targets are implicated in diseases and should be used for subsequent drug development requires the development of not only systematic procedures but also high-throughput approaches for determining which targets are a part of disease relevant pathways are critical to the drug discovery process.

The levels of proteins are determined by the balance between their rates of synthesis and degradation. The ubiquitin-mediated proteolysis is the major pathway for the selective degradation of intracellular proteins. Consequently, selective ubiquitination of a variety of intracellular targets regulates essential cellular functions such as gene expression, cell cycle, signal transduction, biogenesis of ribosomes and DNA repair. Another major function of ubiquitin ligation is to regulate intracellular protein sorting. Whereas poly-ubiquitination targets proteins to proteasome-mediated degradation, attachment of a single ubiquitin molecule (mono-ubiquitination) to proteins regulates endocytosis of cell surface receptors and sorting into lysosomes. It was also demonstrated that ubiquitination controls sorting of proteins in the trans-golgi (TGN).

The linkage of ubiquitin to a substrate protein is generally carried out by three classes of accessory enzymes in a sequential reaction. Ubiquitin activating enzymes (E1) activate ubiquitin by forming a high energy thiol ester intermediate. Activation of the C-terminal Gly of ubiquitin by E1, is followed by the activity of a ubiquitin conjugating enzyme E2 which serves as a carrier of the activated thiol ester form of ubiquitin during the transfer of ubiquitin directly to the third enzyme, E3 ubiquitin protein ligase. E3 ubiquitin protein ligase is responsible for the final step in the conjugation process which results in the formation of an isopeptide bond between the activated Gly residue of ubiquitin, and an .alpha. —NH group of a Lys residue in the substrate or a previously conjugated ubiquitin moiety. See, e.g., Hochstrasser, M., Ubiquitin-Dependent Protein Degradation, Annu. Rev. Genet., 30:405 (1996).

E3 ubiquitin protein ligase, as the final player in the ubiquitination process, is responsible for target specificity of ubiquitin-dependent proteolysis. A number of E3 ubiquitin-protein ligases have previously been identified. See, e.g., D'Andrea, A. D., et al., Nature Genetics, 18:97 (1998); Gonen, H., et al., Isolation, Characterization, and Purification of a Novel Ubiquitin-Protein Ligase, E3-Targeting of Protein Substrates via Multiple and Distinct Recognition Signals and Conjugating Enzymes, J. Biol. Chem., 271:302 (1996). Accordingly, E3 enzymes are potential drug targets and this application provides a systematic method for identifying and validating potential E3 drug targets.

### SUMMARY

In one aspect, the application provides a systematic method of creating a database of related protein or nucleic acid sequences with annotations of the potential disease associations of the sequences; and a method for testing the potential disease associations by means of a biological assay and validating the disease association by either decreasing expression of the sequence of interest or increasing expression of the sequence of interest.

In one aspect, the application provides a method of testing and validating potential drug targets. In one aspect the application provides a method of creating a comprehensive database of related protein and/or nucleic acid sequences; i.e., the protein and nucleic acid sequences are included in the database based upon certain sequence information, structural and/or functional information. In one aspect, the application provides sequences that are sorted based upon sequence, structural, functional, and biological activity. The sequences may be further clustered based upon potential disease association; such as for example, the presence or absence of certain domains may be indicative of potential disease correlations of that protein or nucleic acid sequence. The database further comprises annotations indicating the relevant disease correlations.

The sequences so clustered may be tested for the potential associated disease correlations by means of biological assays. For example, if the associated disease is viral infection, a biological assay may be assaying for the release of virus like particles; if the disease is a proliferative disease the biological assay may be determining the rate of proliferation of the diseased cells. In another aspect, the associated disease may be a ubiquitin-mediated disorder and the assay may determine an aspect of protein degradation, protein trafficking, or cellular localization of proteins. In other embodiments, the assay may be determining any disease characteristic of the associated disease by means of the biological assay.

In another aspect, the application provides methods of validating the disease associations by decreasing the expression of the sequence of interest and determining the effect of such a decrease by means of a biological assay. In one embodiment, if the associated disease is a viral infection, the effect of decreasing expression of the sequence of interest on the release of the virus like particles is determined. Thus, if decreasing the expression of the sequence of interest results in a decrease in the release of the virus like particles the sequence may be a potential drug target for viral infection. Similarly, if decreasing the expression of the sequence of interest results in a decrease in the rate of proliferation of a diseased cell such as a tumor cell the sequence may be a potential drug target for proliferative disorders. Thus, if decreasing the expression alters any disease characteristic of the associated disease, the sequence may be a potential drug target for the associated disease.

In another embodiment, the application provides methods for validating the disease associations by increasing the expression of the sequence of interest. For example, if the sequence of interest is a tumor suppressor increasing expression of the sequence may alter a disease characteristic of an associated disease. In other embodiments, the application provides additional drug targets such as the substrates of various enzymes such as the E3 proteins, wherein either increasing expression of the ligase or decreasing expression of its substrate may alter a disease characteristic of the associated disease. For example, the tumor suppressor von Hippel-Lindau is associated with certain E3-associated diseases; increasing expression of the von Hippel-Lindau gene or decreasing expression of its substrate would alter at least one disease characteristic of the E3 associated disease. Accordingly, in one aspect, the substrate may be a potential drug target for the E3-associated disease.

In one aspect, this invention provides a method of identifying a potential human E3 drug target comprising providing a database comprising human E3 nucleic acid or protein sequences. These sequences are sorted based on their structural and functional attributes providing an E3-associated disease specific database. The potential involvement of E3's in disease is assessed by the criteria which include the following:

1. An E3 that might interact with proteins whose modification by ubiquitin and/or abnormal degradation are the cause for a disease/pathological condition.

2. Potential E3's will be selected from E3's that contain specific structural domains and or motifs that are likely to interact with a specific domains/motifs on the interacting protein.

3. An E3, the cellular localization of which suggests possible interaction with an interacting protein.

4. Abnormal expression of an individual E3 that correlates with a disease/pathological condition.

5. Abnormal activity (due to a mutation or abnormal regulation) of an E3 that is associated with a disease or a pathological condition.

Once the E3 sequences are sorted based upon either their structural attributes or their E3 disease-associations, this invention provides assays for measuring a disease characteristic of said E3-associated disease; for example, such disease characteristics include determining the release of viral like particles from infected cells or cells transfected with plasmids containing a nucleic acid sequence encoding for non infectious viral DNA (e.g. HIV-VLP, VP40 etc′), determining the differential expression of said E3s in a normal cells in comparison to a cell exhibiting at least one symptom of a E3-associated disease etc. Upon identifying a potential E3 target that is implicated in an E3-associated disease, the expression of said E3 is altered, i.e., either increased or decreased to determine whether the change in expression results in a change in the output of the assay.

In another aspect, this invention provides a database comprising human E3 nucleic acid or protein sequences and determining the differential expression of said human E3 in a cell exhibiting disease characteristics of an E3 associated disease and a corresponding normal cell. The expression of said E3 is then altered to determine the effect of decreased E3 expression on said cell exhibiting disease characteristics of an E3 associated disease, wherein a change in said disease characteristics is indicative that said human E3 is a potential drug target for said E3 associated disease.

Identification of potential E3 drug targets provides a means assaying for effective therapeutics.

### BRIEF DESCRIPTION OF THE DRAWINGS

FIG. 1 is a flow-chart of a process for identifying human E3 proteins that may be involved in diseases or other biological processes of interest.

FIG. 2 is a flow-diagram illustrating creation of a database of human E3 proteins.

FIG. 3 provides an exemplary schematic representation of some of the E3-domains present in the E3 proteins.

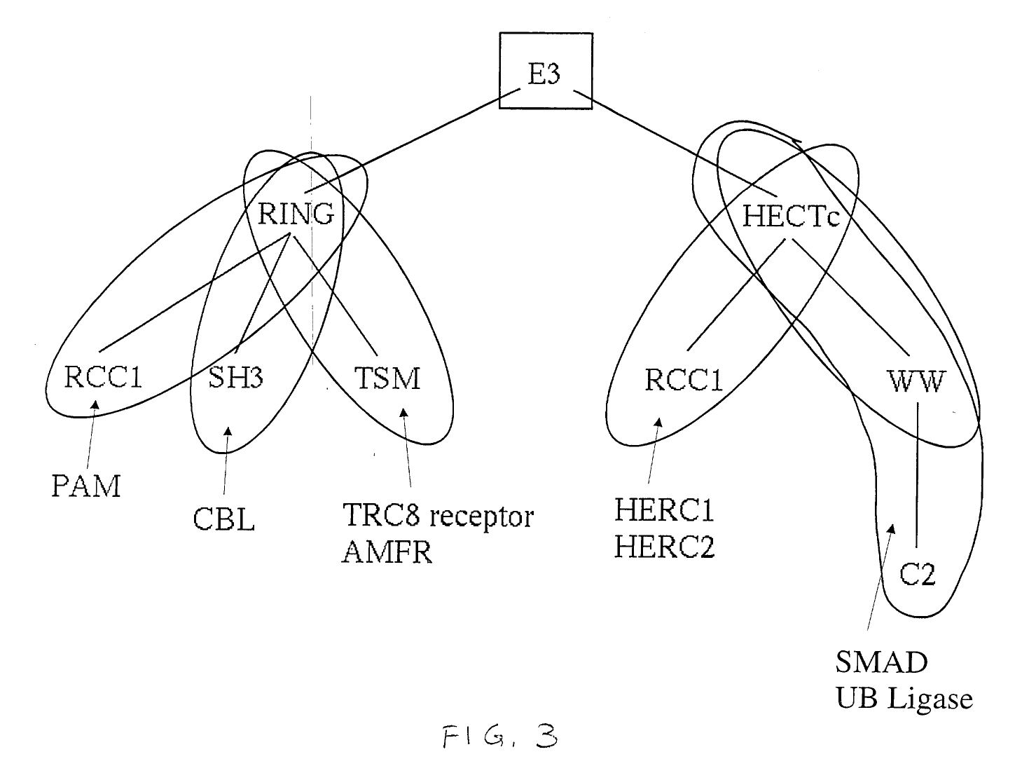

FIG. 4 shows results from a screen to identify E3 proteins that are drug targets for the treatment of HIV and related viruses. A Virus-Like Particle (VLP) 30 Assay was used. The figure shows viral proteins in the cellular fraction (top panel) and in released VLPs (bottom panel). The VLP assay was performed with a wild-type viral p6 protein and a mutant p6 protein as positive and negative controls, respectively. siRNA knockdowns of various mRNAs were tested for effects on VLP production. Knockdown of POSH resulted in complete or near-complete inhibition of VLP production.

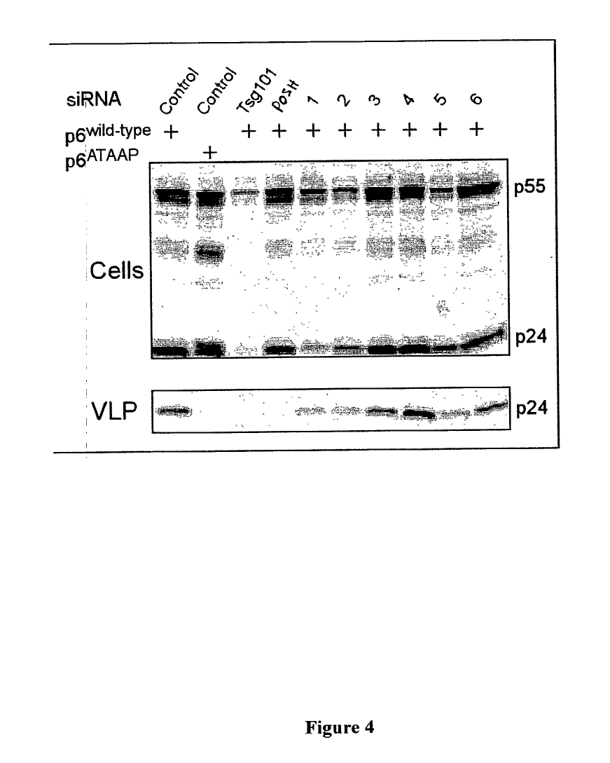

FIG. 5 shows a pulse-chase VLP experiment comparing the kinetics of VLP production in normal (WT) VLP assay conditions and in a POSH knockdown (POSH+WT). siRNA knockdown of POSH results in complete or near-complete inhibition of VLP production.

### DETAILED DESCRIPTION

Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

The phrase “a corresponding normal cell of” or “normal cell corresponding to” or “normal counterpart cell of” a diseased cell refers to a normal cell of the same type as that of the diseased cell. For example, a corresponding normal cell of a B lymphoma cell is a B cell.

An “address” on an array, e.g., a microarray, refers to a location at which an element, e.g., an oligonucleotide, is attached to the solid surface of the array.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

By “array” or “matrix” is meant an arrangement of addressable locations or “addresses” on a device. The locations can be arranged in two dimensional arrays, three dimensional arrays, or, other matrix formats. The number of locations can range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single stranded. Arrays wherein the probes are oligonucleotides are referred to as “oligonucleotide arrays” or “oligonucleotide chips.” A “microarray,” also referred to herein as a “biochip” or “biological chip” is an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance.

The term “associated disease” as used herein refers to a disease that is correlated to a certain nucleic acid or protein sequence because of the presence or absence of certain sequence information, structural or functional information, and/or biological activity of that nucleic acid or protein sequence.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The term “biomarker” of a disease refers to a gene which is up- or down-regulated in a diseased cell of a subject having the disease relative to a counterpart normal cell, which gene is sufficiently specific to the diseased cell that it can be used, optionally with other genes, to identify or detect the disease. Generally, a biomarker is a gene that is characteristic of the disease.

A nucleotide sequence is “complementary” to another nucleotide sequence if each of the bases of the two sequences match, i.e., are capable of forming Watson-Crick base pairs. The term “complementary strand” is used herein interchangeably with the term “complement.” The complement of a nucleic acid strand can be the complement of a coding strand or the complement of a non-coding strand.

The phrases “conserved residue” “or conservative amino acid substitution” refer to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Ile,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, Ile, Met and Cys, and

(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

“Differential gene expression pattern” between cell A and cell B refers to a pattern reflecting the differences in gene expression between cell A and cell B. A differential gene expression pattern can also be obtained between a cell at one time point and a cell at another time point, or between a cell incubated or contacted with a compound and a cell that was not incubated with or contacted with the compound.

The term “domain” as used herein refers to a region within a protein that comprises a particular structure or function different from that of other sections of the molecule.

A “HECT domain” or “HECT” is a protein also known as “HECTC” domain involved in E3 ubiquitin ligase activity. Certain HECT domains are 100-400 amino acids in length and comprise an amino acid sequence as set forth in the following consensus sequence (amino acid nomenclature is as set forth in Table 1):

Pro Xaa3 Thr Cys Xaa2-4 Leu Xaa Leu Pro Xaa Tyr (SEQ ID NO. 1).

E3 as used herein refers to a nucleic acid or encoded protein that is involved with substrate recognition in ubiquitin-mediated proteolysis, in membrane trafficking and protein sorting. Ubiquitin-mediated proteolysis is the major pathway for the selective, controlled degradation of intracellular proteins in eukayotic cells. 30 E3 proteins include one or more of the following exemplary domains and/or motifs:

HECT, RING, F-BOX, U-BOX, PHD, etc.

“E3-associated Disease” refers to any disease wherein: (1) an E3 that interacts with interacting proteins whose modification by ubiquitin and/or abnormal degradation are the cause for a disease/pathological condition; (2) an E3 protein is implicated in interacting with a specific domains/motifs such as a domain of an interacting protein such as the late domain of a viral protein, thereby resulting in viral infectivity; (3) an E3, the cellular localization of which suggests possible interaction with an Interacting protein that may cause a disease or pathological condition; (4) differential expression of an E3 gene and or protein correlates with a disease/pathological condition: and (5) aberrant activity (due to a mutation or abnormal regulation) of an E3 that is associated with a disease or a pathological condition. Exemplary E-associated diseases include but are not limited to viral infections, preferably retroviral infections such as HIV, Ebola, CMV, etc., various cancers such as breast, lung, renal carcinoma, etc., cystic fibrosis, and certain diseases of the CNS such as autosomal recessive juvenile parkinsonism.

A “disease characteristic” as used herein refers any one or more of the following: any phenotype that is distinctive of a disease state or any artificial phenotype that is a proxy for a phenotype that is distinctive of a disease state, or that distinguishes a diseased cell from a normal cell.

“A diseased cell of an associated disease” refers to a cell present in subjects having an associated diseases D, which cell is a modified form of a normal cell and is not present in a subject not having disease D, or which cell is present in significantly higher or lower numbers in subjects having disease D relative to subjects not having disease D. For example, a diseased cell may be a cancerous cell.

“A diseased cell of an E3-associated disease” refers to a cell present in subjects having an E3-associated diseases D′; which &ell is a modifiied from of a normal cell and is not present in a subject not having disease D′, or which cell is present in significantly higher or lower numbers in subjects having disease D′ relative to subjects not having disease D′. For example, a diseased cell may be a cell infected with a virus or a cancerous cell.

The term “drug target” refers to any gene or gene product (e.g. RNA or polypeptide) with implications in an associated disease or disorder. Examples include various proteins such as enzymes, oncogenes and their polypeptide products, and cell cycle regulatory genes and their polypeptide products. In one aspect, the drug target may be an E3.

The term “expression profile,” which is used interchangeably herein with “gene expression profile” and “finger print” of a cell refers to a set of values representing mRNA levels of 20 or more genes in a cell. An expression profile preferably comprises values representing expression levels of at least about 30 genes, preferably at least about 50, 100, 200 or more genes. Expression profiles preferably comprise an mRNA level of a gene which is expressed at similar levels in multiple cells and conditions, e.g., GAPDH. For example, an expression profile of a diseased cell of an E3-associated disease D′ refers to a set of values representing mRNA levels of 20 or more genes in a diseased cell.

The term “heterozygote,” as used herein, refers to an individual with different alleles at corresponding loci on homologous chromosomes. Accordingly, the term “heterozygous,” as used herein, describes an individual or strain having different allelic genes at one or more paired loci on homologous chromosomes.

The term “homozygote,” as used herein, refers to an individual with the same allele at corresponding loci on homologous chromosomes. Accordingly, the term “homozygous,” as used herein, describes an individual or a strain having identical allelic genes at one or more paired loci on homologous chromosomes.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes which contain certain mismatches, provided that the two strands are still forming a double stranded helix. “Stringent hybridization conditions” refers to hybridization conditions resulting in essentially specific hybridization.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “Interacting Protein” refers to protein capable of interacting, binding, and/or otherwise associating to a protein of interest, such as for example a human E3 protein. Examples of these proteins include for example the “Late domain” or “L domain”, which is a small portion of a Gag protein that promotes efficient release of virion particles from the membrane of the host cell. L domains typically comprise one or more short motifs (L motifs). Exemplary sequences include: PTAPPEE, PTAPPEY, P(T/S)AP, PxxL, PPxY (eg. PPPY), YxxL (eg. YPDL), PxxP.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the terms “label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens) and the like. The term “fluorescer” refers to a substance or a portion thereof which is capable of exhibiting fluorescence in the detectable range. Particular examples of labels which may be used under the invention include fluorescein, rhodamine, dansyl, umbelliferone, Texas red, luminol, NADPH, alpha -beta -galactosidase and horseradish peroxidase.

The “level of expression of a gene in a cell” refers to the level of mRNA, as well as pre-mRNA nascent transcript(s), transcript processing intermediates, mature mRNA(s) and degradation products, encoded by the gene in the cell.

The phrase “normalizing expression of a gene” in a diseased cell refers to a means for compensating for the altered expression of the gene in the diseased cell, so that it is essentially expressed at the same level as in the corresponding non diseased cell. For example, where the gene is over-expressed in the diseased cell, normalization of its expression in the diseased cell refers to treating the diseased cell in such a way that its expression becomes essentially the same as the expression in the counterpart normal cell. “Normalization” preferably brings the level of expression to within approximately a 50% difference in expression, more preferably to within approximately a 25%, and even more preferably 10% difference in expression. The required level of closeness in expression will depend on the particular gene, and can be determined as described herein.

The phrase “normalizing gene expression in a diseased cell” refers to a means for normalizing the expression of essentially all genes in the diseased cell.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including Hidden Markov Model (HMM), FASTA and BLAST. HMM, FASTA and BLAST are available through the National Center for Biotechnology Information, National Library of Medicine, National Institutes of Health, Bethesda, Md. and the European Bioinformatic Institute EBI. In one embodiment, the percent identity of two sequences can be determined by these GCG programs with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. More techniques and algorithms including use of the HMM are describe in Sequence, Structure, and Databanks: A Practical Approach (2000), ed. Oxford University Press, Incorporated. In Bioinformatics: Databases and Systems (1999) ed. Kluwer Academic Publishers. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. A mismatch in a duplex between a target polynucleotide and an oligonucleotide or olynucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a basepair of the perfectly matched duplex.

As used herein, a nucleic acid or other molecule attached to an array, is referred to as a “probe” or “capture probe.” When an array contains several probes corresponding to one gene, these probes are referred to as “gene-probe set.” A gene-probe set can consist of, e.g., 2 to 10 probes, preferably from 2 to 5 probes and most preferably about 5 probes.

The “profile” of a cell's biological state refers to the levels of various constituents of a cell that are known to change in response to drug treatments and other perturbations of the cell's biological state. Constituents of a cell include levels of RNA, levels of protein abundances, or protein activity levels.

The term “protein” is used interchangeably herein with the terms “peptide” and “polypeptide.”

An expression profile in one cell is “similar” to an expression profile in another cell when the level of expression of the genes in the two profiles are sufficiently similar that the similarity is indicative of a common characteristic, e.g., being one and the same type of cell. Accordingly, the expression profiles of a first cell and a second cell are similar when at least 75% of the genes that are expressed in the first cell are expressed in the second cellat a level that is within a factor of two relative to the first cell.

An “RCC1 domain” is a domain that interacts with small GTPases to promote loss of GDP and binding of GTP. Certain RCC1 domains are about 50-60 amino acids in length. Often RCC1 domains are found in a series of repeats. The first RCC1 domain was identified in a protein called “Regulator of Chromosome Condensation” (RCC1), which interacts with the small GTPase Ran. In the RCC1 protein, a series of seven tandem repeats of a domain of about 50-60 amino acids fold to form a beta-propeller structure (Renault et al. Nature 1998 392:9-101). RCC1 domains are known to interact with other types of small GTPases including members of the Arf, Rab, Rac and Rho families.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain”, “Ring Finger” or “RING” is a zinc-binding domain also known as “ZF-C2HC4” with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys (SEQ ID NO: 2) or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys (SEQ ID NO: 3). Preferred RING domains of the invention bind to various protein partners to form a complex that has ubiquitin ligase activity. RING domains preferably interact with at least one of the following protein types: F box proteins, E2 ubiquitin conjugating enzymes and cullins.

The term “RNA interference”, “RNAi” or “siRNA” are all refers to any method by which expression of a gene or gene product is decreased by introducing into a-target cell one or more double-stranded RNAs which are homologous to the gene of interest (particularly to the messenger RNA of the gene of interest).

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the polypeptide is disrupted.

As used herein, the term “transgene” means a nucleic acid sequence (encoding, e.g., one of the target nucleic acids, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

The term “treating” a disease in a subject or “treating” a subject having a disease refers to subjecting the subject to a pharmaceutical treatment, e.g., the administration of a drug, such that at least one symptom of the disease is decreased.

The term “Ubiquitin-mediated disorder” as used herein refers to a disorder resulting from an abnormal Ubiquitin-mediated cellular process such as for example ubiquitin-mediated degradation, protein trafficking, and or protein sorting.

The term “Unigene” or “unigene cluster” refers to an experimental system for automatically partitioning Genbank sequences into a non-redundant set of Unigene clusters. Each Unigene cluster contains sequences that represent a unique gene, as well as related information such as the tissue types in which the gene has been expressed and map location. In addition, to well characterized genes, EST sequences are also included in these clusters. Such clusters may be downloaded from ftp://ncbi.nlm.nih.gov/repository/Unigene/.

The phrase “value representing the level of expression of a gene” refers to a raw number which reflects the mRNA level of a particular gene in a cell or biological sample, e.g., obtained from experiments for measuring RNA levels.

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “variant,” when used in the context of a polynucleotide sequence, may encompass a polynucleotide sequence related to that of gene X or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

A “WW Domain” is a small functional domain found in a large number of proteins from a variety of species including humans, nematodes, and yeast. WW domains are approximately 30 to 40 amino acids in length. Certain WW domains 30 may be defined by the following consensus sequence (Andre and Springael, 1994, Biochem. Biophys. Res. Comm. 205:1201-1205) (amino acid nomenclature is as set forth in Table 1): Trp Xaa₆₋₉ Gly Xaa₁₋₃ X4 X4 Xaa₄₋₆ X1 X8 Trp Xaa₂ Pro (SEQ ID NO: 4). In certain instances a WW domain will be flanked by stretches of amino acids rich in histidine or cysteine. In some cases, the amino acids in the center of WW domains are quite hydrophobic. Preferred WW domains bind to the L domains of retroviral Gag proteins. Particularly preferred WW domains bind to an amino acid sequence of ProProXaaTyr (SEQ ID NO: 5).

TABLE 1
Abbreviations for classes of amino acids*

| | | Amino Acids |
|---|---|---|
| Symbol | Category | Represented |
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, |
| | | Met, Asn, Pro, Gln, Arg, |
| | | Ser, Thr, Val, Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, |
| | | Ile, Lys, Leu, Met, Thr, |
| | | Val, Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.embl-heidelberg.de/SMART_DATA/alignments/consensus/grouping.html.

Creating a Database

In one aspect the application provides a method of creating a comprehensive database of related protein and/or nucleic acids; i.e., the protein and nucleic acid sequences are included in the database based upon certain sequence information, structural and/or functional information. In one aspect, the application provides sequences that are sorted based upon sequence, structural, functional, and biological activity. The sequences may be further clustered based upon potential disease association; such as for example, the presence or absence of certain domains may be indicative of potential disease correlations of that protein or nucleic acid sequence. The database further comprises annotations indicating the relevant disease correlations. In an illustrative example, the application provides method for creating an E3 database.

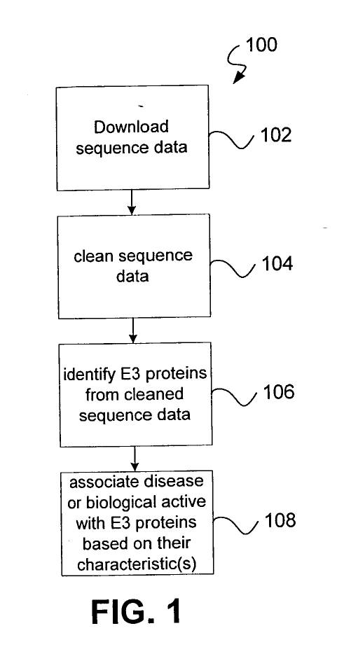

FIG. 1 illustrates a process 100 that identifies human E3 proteins and/or nucleic acid sequences that may be involved in diseases or other biological processes of interest. As shown, the process operates on data describing human protein or nucleic acid sequences. Such data may be downloaded 102 from a variety of sources such as the publicly available NCBI (National Center for Biotechnology Information) or Swiss Prot databases or from proprietary databases such as for examples the databases owned by Incyte Inc. or Celera Inc. Publicly available databases include for example, the NCBI database of human protein sequences on the World Wide Web at http://www.ncbi.nlm.nih.gov/Entrez/batch.html. and the EBI.

As shown, the process 100 may clean 104 the sequences to identify human protein sequences. For example, the process 100 may eliminate redundant sequence information. The process 100 may also eliminate sequence portions based on the polypeptide length. For instance, the process 100 may eliminate polypeptides less than some specified length of amino acids (e.g., 10 or 20) or between a range of lengths (e.g., 25-30).

The process 100 then identifies 106 which sequences correspond to human E3 protein sequences. For example, the process 100 may determine whether a particular sequence exhibits one or more domains associated with E3 proteins. A domain is a recurring sequence pattern or motif. Generally, these domains have a distinct evolutionary origin and function. In particular, the human E3 proteins can include HECT, Ubox, RING, PHD, and/or fbox domains. Based on either the domains present or other characteristics, the process 100 can associate 108 a disease or other biological activity with the E3 proteins. The E3 proteins are identified as having at least a HECT, RING, Ubox, Fbox, ZN3 or PHD domain. In certain embodiments the E3 proteins are identified as having at least a HECT or RING domain.

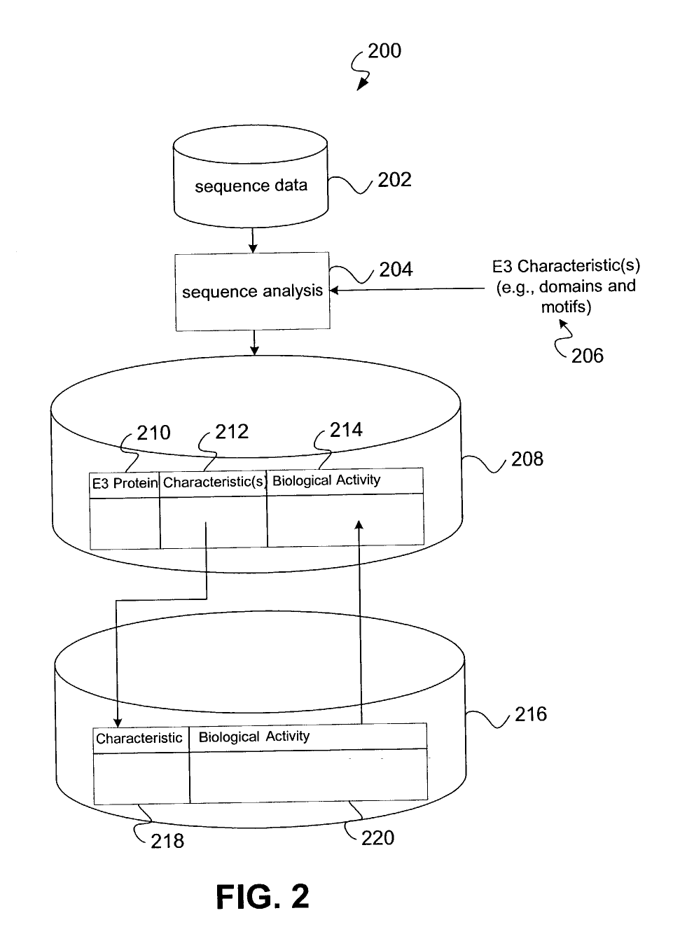

FIG. 2 illustrates a sample implementation 200 of this process in greater detail. As shown, the implementation 200 includes a database 202 of sequence data. Again, the database 202 may be assembled or downloaded from a variety of sources such as the National Institute of Health's (NIH) human genome databases or the EBI human genome databases. Instead of, or in addition to, protein sequences, the database 202 may also include nucleotide and/or gene sequences associated with particular proteins. The database 202 may also include sequence annotations.

Sequence analysis software 204 can identify E3 characteristics 206 indicated by the sequences. Such characteristics 206 can include domains and motifs such as RING, HECT, Ubox, Fbox, PHD domains or the PTA/SP motif. For example, the software can search for consensus sequences of particular domains/motifs. The consensus sequences for some of these exemplary motifs are set forth in the definition section provided above.

The sequence analysis software 204 discussed above may include a number of different tools. For example, the CD-Search Service provided by NCBI. This service provides a useful method of identifying conserved domains that might be present in a protein sequence. The CDD (conserved domain database) contains domains derived from two collections, Smart and Pfam. In particular, Smart (Simple Modular Architecture Research Tool) is a web-based tool for studying such domains (http://SMART.embl-heidelberg.de). It includes more than 400 domain families found in signaling, extracellular, and chromatin-associated proteins. These domains are extensively annotated with respect to phyletic distributions, functional class, tertiary structures, and functionally important residues. Similarly, Pfam (http://pfam.wustl.edu) is a large collection of multiple sequence alignments and hidden Markov models covering common protein domains. As of August 2001, Pfam contains alignments and models for 3071 protein families.

The sequence analysis software 204 may be independently developed. Alternatively, public software may be used. For example, the process may use the Reverse Position-Specific (RPS) Blast (Basic Local Alignment Search Tool) tool. In this algorithm, a query sequence is compared to a position-specific score matrix prepared from the underlying conserved domain alignment. Hits are displayed as a pair-wise alignment of the query sequence with a representative domain sequence, or as a multiple alignment.

The characteristics 206 may also include unigene clusters. Each human E3 protein is then compared to the downloaded clusters to determine the particular cluster that it belongs to. Once the E3 protein has been matched to a cluster we determine what other proteins belong to this cluster and introduce these into the E3 database.

As shown, analysis 204 of the sequence data 202 yields a comprehensive list of E3 proteins and other related proteins 210. Such information may be organized in a database 208 such as a relational database. The database 208 may also store characteristics 212 of the different proteins such as the presence or absence of domains such as WW, RCCI, C2, Cue, SH3, SH2, and even Ubox, fbox, RING, HECT and PHD themselves. Based on these characteristics 212, software can associate the protein 210 with a disorder, disease, or other biological activity. For example, the software may access a database 216 associating different protein characteristics 218 with different biological activities 220. Needless to say, the database 208 may be constantly updated to include either new proteins 210, or other associated characteristics 212 and biological activity 220.

As can be seen from this discussion, databases comprising related sequences may be created by sorting the protein and nucleic acid sequences based on structural, functional and biological activity. As such, the related sequences may be examined for particular domains or motifs and then further clustered based on potential correlations with various associated diseases.

Biological Assays

In one aspect, the application provides methods for determining or testing whether a particular sequence may be correlated to an associated disease. In one embodiment, this application provides a means for determining whether a particular gene or encoded protein, such as an E3 gene or the encoded human E3 protein, is involved in a disease or other biological process of interest. In one aspect, the application provides functional biological assays for correlating protein and nucleic acid sequences with associated diseases or pathological conditions.

The potential involvement of a protein such as a human E3 protein in a disease or biological process of interest may be assessed using a number of methods that are known to the skilled artisan. Some exemplary methods for assessing disease correlations or the involvement of proteins in a biological process of interest, include:

I. Interaction of the proteins such as the human E3 proteins with specific domains or motifs of an Interacting Protein. It is believed that in the course of normal activities the E3 proteins will be free in the cytoplasm or associated with an intracellular organelle, such as the nucleus, the Golgi network, etc. However, during a viral infection, it is possible that certain host proteins, such as certain E3 proteins may be recruited to the cell membrane to participate in viral maturation, including ubiquitination and membrane fusion. For example, the human E3 proteins containing a HECT domain, a RING domain, and a WW or SH3 domain interact with the viral proteins such as the gag protein. In one aspect, the WW domain of the E3 proteins interacts with the late domain of the gag protein having the consensus sequence PxxY. Therefore, E3 proteins having such domains may mediate the ubiquitination of gag to facilitate viral maturation, and as such may be potential drug targets for treating viral infections, such as retroviral infections.

In a further aspect the application provides diagnostic assays for determining whether a cell is infected with a virus and for characterizing the nature, progression and/or infectivity of the infection. As a result, the detection of a E3 protein associated with the plasma membrane fraction may be indicative of a viral infection. Additionally, the presence of E3 proteins at the plasma membrane may also suggest that the infective virus is in the process of reproducing and is therefore actively engaged in infective or lytic activity (versus a lysogenic or otherwise dormant activity).

A number of assays may be useful in studying the potential interaction of human host proteins with viral interacting proteins. For example, such an assay could involve the detection of virus like particles from cells transfected with a virus or cells infected with a virus, such as a retrovirus.

Association of the proteins of the invention, such as the E3 proteins with the plasma membrane may be detected using a variety of techniques known in the art. For example, membrane preparations may be prepared by breaking open the cells (via sonication or detergent lysis) and then separating the membrane components from the cytosolic fraction via centrifugation. Segregation of proteins into the membrane fraction can be detected with antibodies specific for the protein of interest using western blot analysis or ELISA techniques. Plasma membranes may be separated from intracellular membranes on the basis of density using density gradient centrifugation. Alternatively, plasma membranes may be obtained by chemically or enzymatically modifying the surface of the cell and affinity purifying the plasma membrane by selectively binding the modifications. An exemplary modification includes non-specific biotinylation of proteins at the cell surface. Plasma membranes may also be selected for by affinity purifying for abundant plasma membrane proteins.

Transmembrane proteins, such as the E3 proteins containing an extracellular domain can be detected using FACS analysis. For FACS analysis, whole cells are incubated with a fluorescently labeled antibody (e.g., an FITC-labelled antibody) capable of recognizinigthe extracellular domain of the protein of interest. The level of fluorescent staining of the cells may then be determined by FACS analyses (see e.g., Weiss and Stobo, (1984) J. Exp. Med., 160:1284-1299). Such proteins are expected to reside on intracellular membranes in uninfected cells and the plasma membrane in infected cells. FACS analysis would fail to detect an extracellular domain unless the protein is present at the plasma membrane.

Localization of the proteins of interest, such as for example the E3 proteins of the invention may also be determined using histochemical techniques. For example, cells may be fixed and stained with a fluorescently labeled antibody specific for the protein of interest. The stained cells may then be examined under the microscope to determine the subcellular localization of the antibody bound proteins.

II. Potential drug target proteins may also be identified on the basis of an interaction with an interacting protein that may be modified by ubiquitin or may undergo abnormal degradation in disease cells, in comparison with normal cells. For example, it is expected that a number of diseases are related to abnormal protein folding and/or protein aggregate formation. In these cases, the abnormally processed protein may be identified, and a drug target such as an E3s drug target may be identified on the basis of an interaction therewith. Interactions may be identified bioinformatically, using, for example, proteome interaction databases that are generated in a variety of ways (high throughput immunoprecipitations, high throughput two-hybrid analysis, etc.). Various databases include information culled from the literature relating to protein function, and such information may also be used to identify drug target E3s that interact with an abnormally processed protein. Interactions may also be determined de novo, using techniques such as those mentioned above. Once a potential drug target such as an E3 is identified, a number of assays may be used for testing its biological effects.

In one example, the abnormally ubiquitinated, degraded or aggregated protein is monitored for ubiquitination, degradation or aggregation in response to a manipulation in activity of the candidate drug target. For example, ubiquitination has been implicated in the turnover of the tumor supressor protein, p53, and other cell cycle regulators such as cyclin A and cyclin B, the kinase c-mos, and various transcription factors such as c-jun, c-fos, and I.kappa B/NF kappa.B. Altering the half-lives of these cellular proteins is expected to have great therapeutic potential, particularly in the areas of autoimmune disease, inflammation, cancer, as well as other proliferative disorders. Rolfe, M., et al., The Ubiquitin-Mediated Proteolytic Pathway as a Therapeutic Area, J. Mol. Med., 75:5 (1997). Many assays described herein and, in view of this application, known to one of skill in the art may be used to test the biological effects of the potential drug target such as the E3s.

III. Potential drug target proteins such as the E3 proteins may be selected on the basis of cellular localization. In a variety of disease states, a cellular dysfunction can be traced to one or more cellular compartments. A protein such as an E3 that localizes to that compartment may be implicated in the disease, particularly where a dysfunctional protein appears to interact with the ubiquitination system. For example, Cystic Fibrosis is an inherited disorder that is linked to reduced surface expression of the Cystic Fibrosis Transduction Regulator (CFTR). Nearly 70% of the affected patients are homozygous for the CFTR AF^{Δ508} mutation. Mutant CFTR is rapidly degraded in the endoplasmic reticulum (ER) via the ubiquitin proteolytic system resulting in reduced surface expression. It is known that modulation of ER-associated protein degradation triggers the Unfolded Protein Response (UPR) which results in the production of a number of proteins that mediate protein folding. The combination of decreased ubiquitination and increased protein folding are expected cause a greater proportion of proteins to successfully mature (Travers et al. (2000) Cell 101:249-258). Accordingly, human E3 proteins that are either known as being localized to the ER or that are integral membrane E3 proteins may mediate the degradation of the mutant CFTR and as such may be potential drug targets for treating cystic fibrosis.

Protein localization such as localization of the E3 may be determined or predicted by bioinformatic analysis, e.g. through examination of protein localization signals present in the amino acid sequences of the E3s present in a database. Exemplary localization signals include signal peptides (indicating that the protein is routed into the ER-mediated secretion pathway), retention sequences, indicating retention atone or more positions in the secretory pathway, such as the ER, a Dart of the Golgi, etc., nuclear localization signals, membrane domains, lipid modification sequences, etc. In view of this specification, one of skill in the art will be able to identify numerous types of sequence information that are indicative of protein localization. In another variant, localization may be determined directly by expression of E3s in a cell line, preferably a mammalian cell line. The protein may be expressed as a native protein, wherein localization would typically be determined by immunofluorescence micorscopy. Alternatively, the protein may be expressed with a detectable tag, such as a fluorescent protein (e.g. GFP, BFP, RFP, etc.), and the localization may be determined by direct immunofluorescence microscopy. Localization may also be determined by cellular fractionation followed by high-throughput protein identification, such as by coupled two-dimensional electrophoresis and mass spectroscopy. This would permit rapid identification of proteins present in various cellular compartments.

Having identified one or more drug target E3 proteins, a number of different assays are available to test the role of the E3 in the disease state. For example, in numerous diseases, a membrane protein is not properly processed and partitioned to the plasma membrane. Accordingly, E3 function may be manipulated (see below) and the level of membrane protein arriving at the membrane measured. Increased delivery of protein to the membrane in response to manipulation of E3 function indicates that the E3 is a valid target for disease therapeutics. As noted above, CFTR maturation is perturbed in cystic fibrosis. In one example, E3s are validated by manipulating the subject E3 and determining the level of mutant CFTR AF^{Δ508} accumulated at the plasma membrane. Likewise, 98% of the erythropoietin receptor fails to mature and is degraded in the secretory pathway. An increased yield of erythropoietin receptor may mimic the effects of erythropoietin itself, which is clinically important stimulator of hematopoiesis. Accordingly, an E3 may be validated by assessing the effect of increasing or decreasing its activity on the amount of erythropoietin at the cell surface.

In further examples, a variety of E3 enzymes may interact with viral proteins that affect the degradation of host proteins passing through the ER. Many viruses co-opt the ER-associated protein degradation pathway to destabilize host proteins that are unfavorable to viral infection. For example, human cytomegalovirus (HCMV) evades the immune system in part by causing the destruction of MHC class I heavy chains. Two HCMV proteins, US11 and US2 cause rapid retrograde transport of the MHC class I heavy chains from the ER to the cytosol, where they are degraded by the proteasome. This process is ubiquitin-dependent. In addition, the HIV virus targets the host CD4 protein for destruction through an ER-associated, ubiquitin-dependent protein degradation pathway. Destruction of CD4 is important because CD4 in the ER associates with and inhibits the maturation of the HIV glycoprotein gp160. Therefore, E3s may be validated, for example, by assessing effects on the processing or localization of MHC class I heavy chains (or other MHC class I complexes) or CD4.

IV. Potential drug targets may also be identified by the differential expression of certain nucleic acids or proteins in disease cells in comparison to normal cells.

In one aspect, differential expression of a protein in a normal cell in comparison with diseased cells, such as a cell manifesting an associated disease, is indicative that the differentially expressed gene may be involved in the associated disease or other biological process. For example, differential expression of an E3 protein in a tumor tissue in comparison with normal tissue may be indicative that the E3 may be involved in tumorigenesis.

In one embodiment, the invention is based on the gene expression profile of cells from an E-3associated disease. Diseased cells may have genes that are expressed at higher levels (i.e., which are up-regulated) and/or genes that are expressed at lower levels (i.e., which are down-regulated) relative to normal cells that do not have any symptoms of the E3-assocaited disease. In particular, certain E3 genes may be up-regulated by at least about 1 fold, preferably 2 fold, more preferably 5 fold, in the diseased cell as compared to the normal cell. Alternatively, certain E3 genes may be down-regulated by at least about 1 fold, preferably 2 fold, more preferably 5 fold in the diseased cells relative to the corresponding normal cells.

Preferred methods comprise determining the level of expression of one or more E3 genes in diseased cells in comparison to the corresponding normal cells. Methods for determining the expression of tens, hundreds or thousands of genes, in diseased cells relative to the corresponding normal cells include, for e.g., using microarray technology. The expression levels of the E3 genes are then compared to the expression levels of the same E3 genes one or more other cell, e.g., a normal cell.

Comparison of the expression levels can be performed visually. In a preferred embodiment, the comparison is performed by a computer.

In another embodiment, values representing expression levels of genes characteristic of an E3 associated disease are entered into a computer system, comprising one or more databases with reference expression levels obtained from more than one cell. For example, the computer comprises expression data of diseased and normal cells. Instructions are provided to the computer, and the computer is capable of comparing the data entered with the data in the computer to determine whether the data entered is more similar to that of a normal cell or of a diseased cell.

In one embodiment, the invention provides a method for determining the level of expression of one or more E3 genes which are up- or down-regulated in a particular E3-associated diseased cell and comparing these levels of expression with the levels of expression of the E3 genes in a diseased cell from a subject known to have the disease, such that a similar level of expression of the genes is indicative that the E3 gene may be implicated in the disease.

Comparison of the expression levels of one or more E3 genes involved with an E3-associated disease with reference expression levels, e.g., expression levels in diseased cells of or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In one embodiment, the invention provides a computer readable form of the E3 gene expression profile data of the invention, or of values corresponding to the level of expression of at least one E3 gene implicated in an E3-associated disease in a diseased cell. The values can be mRNA expression levels obtained from experiments, e.g., microarray analysis. The values can also be mRNA levels normalized relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data can be in the form of a table, such as an Excel table. The data can be alone, or it can be part of a larger database, e.g., comprising other expression profiles. For example, the expression profile data of the invention can be part of a public database. The computer readable form can be in a computer. In another embodiment, the invention provides a computer displaying the gene expression profile data.

In one embodiment, the invention provides a method for determining the similarity between the level of expression of one or more E3 genes characteristic of an E3 associated disease in a first cell, e.g., a cell of a subject, and that in a second cell, comprising obtaining the level of expression of one or more genes characteristic of E3 associated disease in a first cell and entering these values into a computer comprising a database including records comprising values corresponding to levels of expression of one or more genes characteristic of said E3 associated disease in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes characteristic of an E3-associated disease D in a query cell with a database including records comprising reference expression or expression profile data of one or more reference cells and an annotation of the type of cell; and (ii) indicating to which cell the query cell is most similar based on similarities of expression profiles. The reference cells can be cells from subjects at different stages of the E3-associated disease.

The relative abundance of an mRNA in two biological samples can be scored as a perturbation and its magnitude determined (i.e., the abundance is different in the two sources of MRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations can be used by a computer for calculating and expression comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods described herein may be supported by and executed on any suitable platform, including commercially available hardware systems, such as IBM-compatible personal computers executing a variety of the UNIX operating systems, such as Linux or BSD, or any suitable operating system such as MS-DOS or Microsoft Windows. In one embodiment, the data processor may be a MIPS R10000, based mullet-processor Silicon-Graphic Challenge server, running IRJX 6.2. Alternatively and optionally, the systems and methods described herein may be realized as embedded programmable data processing systems that implement the processes of the invention. For example, the data processing system can comprise a single board computer system that has been integrated into a piece of laboratory equipment for performing the data analysis described above. The single board computer (SBC) system can be any suitable SBC, including the SBCs sold by the Micro/Sys Company, which include microprocessors, data memory and program memory, as well as expandable bus configurations and an on-board operating system.

Optionally, the data processing systems may comprise an Intel Pentium®-based processor or AMD processor or their equals of adequate clock rate and with adequate main memory, as known to those skilled in the art. Optional external components may include a mass storage system, which can be one or more hard disks (which are typically packaged together with the processor and memory), tape drives, CDROMS devices, storage area networks, or other devices. Other external components include a user interface device, which can be a monitor, together with an input device, which can be a “mouse” ,or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems. The network can be, for example, an NFS network with a Postgres SQL relational database engine and a web server, such as the Apache web server engine. However, the server may be any suitable server process including any HTTP server process including the Apache server. Suitable servers are known in the art and are described in Jamsa, Internet Programming, Jamsa Press (1995), the teachings of which are herein incorporated by reference. Accordingly, it shall be understood that in certain embodiments, the systems and methods described herein may be implemented as web-based systems and services that allow for network access, and remote access. To this end, the server may communicate with clients stations. Each of the client stations can be a conventional personal computer system, such as a PC compatible computer system that is equipped with a client process that can operate as a browser, such as the Netscape Navigator browser process, the Microsoft Explorer browser process, or any other conventional or proprietary browser process that allows the client station to download computer files, such as web pages, from the server.

In certain embodiments the systems and methods described herein are realized as software systems that comprise one or more software components that can load into memory during operation. These software components collectively cause the computer system to function according to the methods of this invention. In such embodiments, the systems may be implemented as a C language computer program, or a computer program written in any high level language including C++, Fortran, Java or BASIC. Additionally, in an embodiment where SBCs are employed, the systems and methods may be realized as a computer program written in microcode or written in a high level language and compiled down to microcode that can be executed on the platform employed. The development of such systems is known to those of skill in the art, and such techniques are set forth in Digital Signal Processing Applications with the TMS320 Family, Volumes I, II, and III, Texas Instruments (1990). Additionally, general techniques for high level programming are known, and set forth in, for example, Stephen G. Kochan, Programming in C, Hayden Publishing (1983).

Additionally, in certain embodiments, these software components may be programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes characteristic of am E3 associated disease. The database may contain one or more expression profiles of genes characteristic of the E3 associated disease in different cells.

The database employed may be any suitable database system, including the commercially available Microsoft Access database, Postgre SQL database system, MySQL database systems, and optionally can be a local or distributed database system. The design and development of suitable database systems are described in McGovern et al., A Guide To Sybase and SQL Server, Addison-Wesley (1993). The database can be supported by any suitable persistent data memory, such as a hard disk drive, RAID system, tape drive system, floppy diskette, or any other suitable system. The system 200 depicted in FIG. 2 depicts several separate databases devices. However, it will be understood by those of ordinary skill in the art that in other embodiments the database device can be integrated into a single system.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

Once again, having identified one or more drug target proteins that are differentially expressed in disease cells, a number of different assays are available to test the role of the drug target protein in the disease state.

For instance, if a E3 protein is identified as being over-expressed in a particular tumor-type, the skilled artisan can readily test for the role of the E3 by conducting a number of assays, for example one could use techniques such as antisense constructs, RNAi constructs, DNA enzymes etc. to decrease the expression of the E3 in a tumor cell line to determine whether inhibition of the E3 results in decreased proliferation. In other embodiments the activity of the E# may be decreased by using techniques such as dominant negative mutants, small molecules, antibodies etc. Other techniques include proliferation assays such as determining thymidine incorporation.

V. Aberrant activity of certain human drug target proteins may also be associated with a disease state or pathological condition.

For example, the association of the E3 proteins with certain disease or disorders provides a disease specific database containing human E3 proteins that may be implicated in the disease or disorder.

Validating Potential Drug Targets

In another aspect, this application provides methods for validating the selected proteins, such as the E3 proteins as viable drug targets. In one embodiment, the methods provide for decreasing the expression of the potential drug targets and determining the effects of the reduction of such expression. The expression of the drug targets may be reduced by a number of methods that are known in the art, such as the use of antisense methods, dominant negative mutants, DNA enzymes, RNAi, ribozymes, to name but a few of such methods.

In another embodiment, the methods provide for increasing the expression of the potential drug targets and determining the effects of the increase of such expression.

One aspect of the invention relates to the use of the isolated “antisense” nucleic acids to inhibit expression, e.g., by inhibiting transcription and/or translation of the potential drug target. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes the potential drug target. Alternatively, the antisense construct is an oligonucleotide probe, which is generated ex vivo and which, when introduced into the cell causes inhibition of expression by hybridizing with the mRNA and/or genomic sequences of the potential drug target. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. No. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659- 2668.

With respect to antisense DNA, oligodeoxyribonucleotides derived from the translation initiation site, e.g., between the −10 and +10 regions of the potential drug target, are preferred. Antisense approaches involve the design of oligonucleotides (either DNA or RNA) that are complementary to MRNA encoding the potential drug target. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′,3′ or coding region of mRNA, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood- brain barrier (see, e.g., PCT Publication No. WO89/10134, published Apr. 25, 1988) hybridization-triggered cleavage agents. (See, e.g., Krol et al., 1988, BioTechniques 6:958- 976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6- isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to the coding region of an mRNA sequence can be used, those complementary to the transcribed untranslated region and to the region

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct, which can be introduced directly into the tissue site.

Alternatively, the potential drug target gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Preferred embodiments of the invention make use of materials and methods for effecting repression of one or more target genes by means of RNA interference (RNAi). RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2+,5′ oligoadenylate synthetase (2′,5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of a target gene in a number of different organisms including Caenorhabditiis elegans (see e.g. Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phophoramidites and thymidine phosphoramidite (Proligo, Germany). Synthetic oligonucleotides are preferably deprotected and gel-purified using methods known in the art (see e.g. 'Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilized programs designed to predict the secondary structure of a specified single stranded nucleic acid sequence and allow selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167) . The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art- e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the targeted gene product following sufficient time for turnover of the endogenous pool after new protein synthesis is repressed, and Northern blot analysis to determine the level of existing target mRNA.

Further compositions, methods and applications of RNAi technology are provided in U.S. patent application Nos. 6,278,039, 5,723,750 and 5,244,805, which are incorporated herein by reference.

Ribozyme molecules designed to catalytically cleave the potential drug target mRNA transcripts can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave MRNA at site specific recognition sequences can be used to destroy particular mRNAs, the use of hammerhead ribozymes is preferred. Hammerhead ribozymes cleave mRNAs at locations dictated by flanking regions that form complementary base pairs with the target MRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which, hybridizes to a target RNA sequence whereafter cleavage of the target RNA takes place. The invention encompasses those Cech-type ribozyrnes which target eight base-pair active site sequences.

As in the antisense approach, the ribozymes can be composed of modified oligonucleotides (e.g., for improved stability, targeting, etc.) and should be delivered to cells expressing the potential drug target. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

A further aspect of the invention relates to the use of DNA enzymes to decrease expression of the potential drug targets. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme (shown schematically in FIG. 1) comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No.6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

The present invention is further illustrated by the following examples which should not be construed as limiting in any way. The contents of all cited references including literature references, issued patents, published or non published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); , Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Examples

### Example 1

Method of Creating the Database

The following procedure illustrates one embodiment of creating a database.

1. NCBI protein database is downloaded from NCBI ftp site: ftp.ncbi.nlm.nih.gov

2. Retrieve hum nr: Retrieve all the human sequence in an automatic way from the following url: http://www.ncbi.nlm.nih.vov/Entrez/batch.html. In the HTML form one can specify that all the protein sequences, from Homo Sapiens are to be retrieved.

3. Whether the protein is a human protein is determined by downloading the full nr file from ncbi ftp site, in a fasta format. All the sequences that have the pattern [Homo Sapiens] at the end of the description sentence (i.e. from the first line) are parsed out.

4. Clean sequences: These sequences are then cleaned. Two scripts are run in order to clean the Human nr fasta file. The first script eliminates all the redundant sequences, and leaves all the unique sequences. The second script removes all the short sequences (less then 30 aa).

5. Run RPS-Blast: RPS-Blast is run locally against the CDD database (which contains the Pfam, SMART and LOAD domains). In addition we look for domains in the prosite database. We also look for different features in the sequences: Transmembrane regions (alom2, tmap), signal peptide and other internal domains/features.

6. Find E3 proteins: this search is done automatically. We look for all the proteins that have one or more of the following domains (Hect, Ring, Ubox, Fbox, PHD). These five domains appear in the different databases (pfam, smart and prosite) in different names. In our search we look for these domains in all the different names, in all the databases.

7. Unigene clusters data: We download the clusters (Hs.data file) from the following ur;: ftp://ncbi.nlm.nih.gov/repiositor/UniGene/.

\mathring{8} E3 Vs. Unigene: We look at each E3 protein from the E3 table; to see in which Unigene Cluster it belongs.

\mathring{9} We check which other proteins are in the E3 clusters, which are not E3 proteins, and introduce them in the E3 database.

In addition, multiple sequence alignment may be performed between all the cluster members against the relative genomic piece. In this way we can see the alternative transcripts of the gene.

In particular, RPS-Blast may be run at least twice. In the first run, an E value of 0.01 may be used, and then all the domains may be run against the human nr. In the second run, an E value of 10 may be used , and only the E3 domains (hect, ring, ubox, fbox, phd) are run against the human nr. In this manner the database will have a lower number of false positives, but have a higher sensitivity to the E3 domains.

Further, the E3 database can integrate links to articles, links to patents, annotations of the proteins and other biological information that may be available for the particular protein.

Examples of E3 polypeptides and nucleic acids that may be incorporated into one or more databases are presented in Table 2, appended at the end of the text. Applicants incorporate by reference herein the nucleic acid and amino acid sequences corresponding to the accession numbers provided in Table 2.

### Example 2

Domains and/or Motifs of Interest

A. Protein Domains That may Play a Role in Virus Biogenesis, Maturation and Release

E3—Domain of E3 Ubiguitin-Protein Lizase

RING—

SMART SM0184; RING=RNF, E3 ubiquitin-protein ligase activity is intrinsic to the RING domain of c-Cb1 and is likely to be a general function of this domain; Various RING fingers exhibit binding activity towards E2's, i.e., the ubiquitin-conjugating enzymes (UBC's).

HECTc—

SMARTSMO0119; Pfam PF00632; HECTc=HECT, E3 ubiquitin-protein ligases. Can bind to E2 enzymes. The name HECT comes from ‘Homologous to the E6-AP Carboxyl Terminus’. Proteins containing this domain at the C-terminus include ubiquitin-protein ligase activity, which regulates ubiquitination of CDC25. Ubiquitin-protein ligase accepts ubiquitin from an E2 ubiquitin-conjugating enzyme in the form of a thioester, and then directly transfers the ubiquitin to targeted substrates. A cysteine residue is required for ubiquitin-thiolester formation. Human thyroid receptor interacting protein 12, which also contains this domain, is a component of an ATP-dependent multi-subunit protein that interacts with the ligand binding domain of the thyroid hormone receptor. It could be an E3 ubiquitin-protein ligase. Human ubiquitin-protein ligase E3A interacts with the E6 protein of the cancer-associated human papillomavirus types 16 and 18. The E6/E6-AP complex binds to and targets the P53 tumor-suppressor protein for ubiquitin-mediated proteolysis.

F-BOX—

SMART SM0256; Pfam PF00646; F-BOX=FBOX=F-box=Fbox. The F-box domain was first described as a sequence domain found in cyclin-F that interacts with the protein SKP1. This domain is present in numerous proteins and serves as a link between a target protein and a ubiquitin-conjugating enzyme. The SCF complex (e.g., Skp1-Cullin-F-box) plays a similar role as an E3 ligase in the ubiquitin protein degradation pathway.

U-BOX—

SMART SM0504. The U-box domain is a modified RING finger domain that is without the full complement of Zn2+-binding ligands. It is found in pre-mRNA splicing factor, several hypothetical proteins, and ubiquitin fusion degradation protein 2, where it may be involved in E2-dependent ubiquitination.

PHD—

SMART SM0249. The PHD domain is a C4HC3 zinc-finger-like motif found in nuclear proteins that are thought to be involved in chromatin-mediated transcriptional regulation. The PHD finger motif is reminiscent of, but distinct from the C3HC4 type RING finger. Like the RING finger and the LIM domain, the PHD finger is expected to bind two zinc ions.

B. Protein Domains That May Play a Role in Virus Biogenesis, Maturation and Release in Combination with E3 Ubipuitin-Protein Ligase

RCC1—Domain that Interacts With Small GTPases such ARF1 That Activates AP1 to Polymerize Clathrin

Pfam PF00415; The regulator of chromosome condensation (RCC1) [MEDLINE: 93242659] is a eukaryotic protein which binds to chromatin and interacts with ran, a nuclear GTP-binding protein IPR002041, to promote the loss of bound GDP and the uptake of fresh GTP, thus acting as a guanine-nucleotide dissociation stimulator (GDS). The interaction of RCC1 with ran probably plays an important role in the regulation of gene expression. RCC1, known as PRP20 or SRM1 in yeast, pim1 in fission yeast and BJ1 in Drosophila, is a protein that contains seven tandem repeats of a domain of about 50 to 60 amino acids. As shown in the following schematic representation, the repeats make up the major part of the length of the protein. Outside the repeat region, there is just a small N-terminal domain of about 40 to 50 residues and, in the Drosophila protein only, a C-terminal domain of about 130 residues.

WW—Domain That Interacts With PxxPP Seq. on Gag L-Domain of HIV

SMART SM0456; Pfam PF00397; Also known as the WWP or rsp5 domain. Binds proline-rich polypeptides. The WW domain (also known as rsp5 or WWP) is a short conserved region in a number of unrelated proteins, among them dystrophin, responsible for Duchenne muscular dystrophy. This short domain may be repeated up to four times in some proteins. The WW domain binds to proteins with particular proline-domains, [AP]-P-P-[AP]-Y, and having fourconserved aromatic positions that are generally Trp. The name WW or WWP derives from the presence of these Trp as well as that of a conserved Pro. It is frequently associated with other domains typical for proteins in signal transduction processes. A large variety of proteins containing the WW domain are known. These include; dystrophin, a multidomain cytoskeletal protein; utrophin, a dystrophin-like protein of unknown function; vertebrate YAP protein, substrate of an unknown serine kinase; mouse NEDD-4, involved in the embryonic development and differentiation of the central nervous system; yeast RSP5, similar to NEDD-4 in its molecular organization; rat FE65, a transcription-factor activator expressed preferentially in liver; tobacco DB10 protein and others.

C2—Domain That Interacts With Phospholipids, Inositol Polyphosphates, and Intracellular Proteins

SMART SM0239; Pfam PF00168; Ca2+-binding domain present in phospholipases, protein kinases C, and synaptotamins (among others). Some do not appear to contain Ca2+-binding sites. Particular C2s appear to bind phospholipids, inositol polyphosphates, and intracellular proteins. Unusual occurrence in perforin. Synaptotagmin and PLC C2s are permuted in sequence with respect to N- and C-terminal beta strands. SMART detects C2 domains using one or both of two profiles.

Interpro abstract (IPR000008): Some isozymes of protein kinase C (PKC) is located between the two copies of the C1 domain (that bind phorbol esters and diacylglycerol) and the protein kinase catalytic domain. Regions with significant homology to the C2-domain have been found in many proteins. The C2 domain is thought to be involved in calcium-dependent phospholipid binding. Since domains related to the C2 domain are also found in proteins that do not bind calcium, other putative functions for the C2 domain like e.g. binding to inositol-1,3,4,5-tetraphosphate have been suggested. The 3D structure of the C2 domain of synaptotagmin has been reported the domain forms an eight-stranded beta sandwich constructed around a conserved 4-stranded domain, designated a C2 key. Calcium binds in a cup-shaped depression formed by the N- and C-terminal loops of the C2-key domain.

CUE—Domain That Recruits E2to ER-Membrane Proximity

SMART SM0546; Pfam PF02845; Domain that may be involved in binding ubiquitin-conjugating enzymes (UBCs). CUE domains also occur in two proteins of the IL-1 signal transduction pathway, tollip and TAB2.

SH3 & SH2—

SMART Sm0252; Pfam PF00017; Src homology 2 domains bind phosphotyrosine-containing polypeptides via 2 surface pockets. Specificity is provided via interaction with residues that are distinct from the phosphotyrosine. Only a single occurrence of a SH2 domain has been found in S. cerevisiae. The Src homology 2 (SH2) domain is a protein domain of about 100 amino-acid residues first identified as a conserved sequence region between the oncoproteins Src and Fps. Similar sequences were later found in many other intracellular signal-transducing proteins. SH2 domains function as regulatory modules of intracellular signalling cascades by interacting with high affinity to phosphotyrosine-containing target peptides in a sequence-specific and strictly phosphorylation-dependent manner. They are found in a wide variety of protein contexts e.g., in association with catalytic domains of phospholipase Cy (PLCy) and the nonreceptor protein tyrosine kinases; within structural proteins such as fodrin and tensin; and in a group of small adaptor molecules, i.e Crk and Nck. In many cases, when an SH2 domain is present so too is an SH3 domain, suggesting that their functions are inter-related. The domains are frequently found as repeats in a single protein sequence. The structure of the SH2 domain belongs to the alpha+beta class, its overall shape forming a compact flattened hemisphere. The core structural elements comprise a central hydrophobic anti-parallel beta-sheet, flanked by 2 short alpha-helices. In the v-src oncogene product SH2 domain, the loop between strands 2 and 3 provides many of the binding interactions with the phosphate group of its phosphopeptide ligand, and is hence designated the phosphate binding loop.

The SH3 domain (SMART SM0326) shares 3D similarity with the WW domain, and may bind to PxxPP sequence of the viral gag protein. Src homology 3 (SH3) domains bind to target proteins through sequences containing proline and hydrophobic amino acids. Pro-containing polypeptides may bind to SH3 domains in 2 different binding orientations. The SH3 domain has a characteristic fold which consists of five or six beta-strands arranged as two tightly packed anti-parallel beta sheets. The linker regions may contain short helices.

Protein domain information may be obtained from any of the following websites: SMART (http://smart.embl-heidelberg.de/), Pfam (http://smart.embl-heidelberg.de/), InterPro (http://www.ebi.ac.uk/interpro/scan.html).

### Example 3

Methods for Screening the Biological Activity of the E3 Proteins and Validating the Role of E3's as Potential Drug Targets

A functional biological assay for a disease or a pathological condition is developed in each instance. RNA interference (RNAi) technology or dominant negative forms of candidate E3s or any of the other techniques that are used in the art to inhibit expression of relevant target proteins may be used. The ability of these method to remedy the abnormality that causes a disease/pathological condition validates the role of the specific E3 and its relevance as a potential drug target.

Identification of an E3 Involved in the Ubiquitin-Mediated Viral Release

Experimental evidence supports a model wherein the release of viral like particles (VLP) from infected cells is dependent on ubiquitination of a viral protein such as gag. Ubiquitintaion of gag indicates that a human E3 protein is involved. The gag proteins, such as the late domain, are known to interact with the HECT domain and a WW or SH3 domain of the E3 proteins. Therefore, human E3 proteins that may have wither a HECT or a WW or SH3domain may mediate the ubiquitination of gag to facilitate viral release.

The detection and/or measurement of the release of VLP from cells infected with retroviral infections provide a convenient biological assay.

The inhibition of VLP release by decreasing the expression of the potential drug target validates the potential drug target.

Identification of an E3 Involved in the Ubiquitin-Mediated Degradation of an Interacting Protein

A ubiquitin-protein ligase that mediates the ubiquitination of CFTR is identified. Cystic fibrosis (CF) is an inherited disorder is caused by the malfunction or reduced surface expression of the Cystic Fibrosis Transduction Regulator (CFTR). Approximately 70% of the affected individuals are homozygous to the CFTR^{ΔF508} mutation. Mutant CFTR is rapidly degraded in the endoplasmic reticulum (ER) via the ubiquitin proteolytic system resulting in inhibition of surface expression. An ER-associated E3 is likely to mediate the ubiquitination of CFTR. Accordingly, preferred E3 candidates are those localized to the ER or those that have the CUE domain. Cell surface expression of CFTR^{Δ508} is used as the functional biological assay. Finally, the target is validated by detecting increased surface expression of CFTR^{Δ508} in cells co-expressing a dominant negative form of a candidate E3 or transfected with a specific RNAi derived from a candidate E3.

### Example 4

Identification and Validation of POSH as a Drug Target for Antiviral Agents

An example of the systems disclosed herein was used to successfully identify a drug target for antiviral agents, and especially agents that are effective against HIV and related viruses.

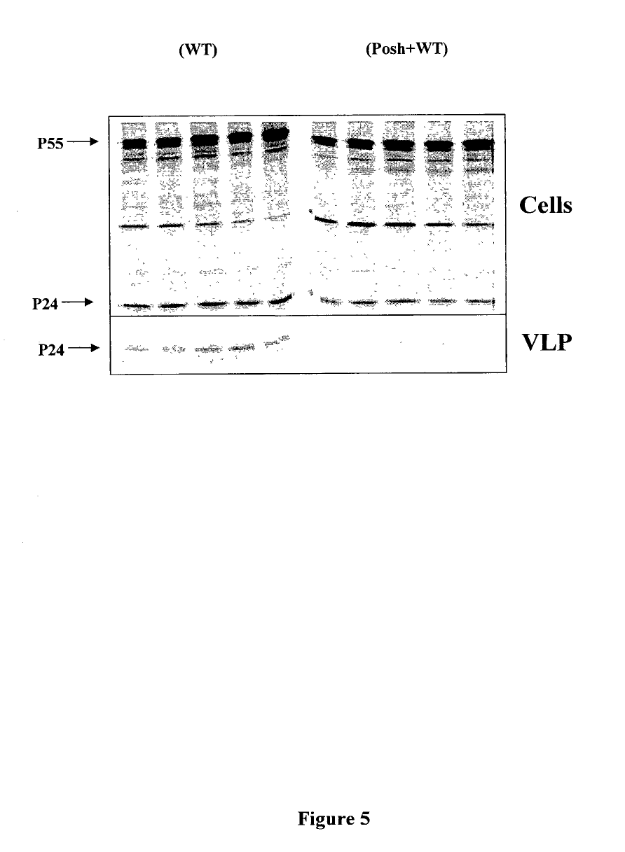

A database of greater than 500 E3 proteins was assembled. The database contained many of the proteins presented in Table 2. A subset of proteins was selected based on various characteristics, such as the presence of RING and SH3 domains or HECT and RCC domains. The proteins of this subset are shown in Table 3. Proteins of the subset were tested for their effects on the lifecycle of HIV using the Virus-Like Particle (VLP) assay system. A knockdown for each protein was created by contacting the assay cells with an siRNA construct specific for an mRNA sequence corresponding to each of the proteins of Table 3. Results for POSH and proteins 1-6 are shown in FIG. 5. Decrease in POSH production by siRNA led to a complete or near-complete disruption of VLP production. A few of the other E3s tested gave partial effects on VLP production, and most E3s had no effect. Tsg101 is used as a positive control.

TABLE 3
E3 subset selected for VLP Assays

| | |
|---|---|
| Gene | Accession |

| | | |
|---|---|---|
| 1. | CEB1 | AB027289 |
| 2. | HERC1 | U50078 |
| 3. | HERC2 | AF071172 |
| 4. | HERC3 | D25215 |
| 5. | ITCH | AF095745 |
| 6. | KIAA1301 | AB037722 |
| 7. | KIAA1593 | AB046813 |
| 8. | Nedd4 | D42055 |
| 9. | NeddL1 | AB048365 |
| 10. | Need4L | AB007899 |
| 11. | PAM | AF07558 |
| 12. | POSH | protlog1 |
| 13. | SMURF1 | AC004893 |
| 14. | SMURF2 | NM_022739 |
| 15. | WWP1 | AL136739 |
| 16. | WWP2 | U96114 |

FIG. 6 shows a pulse-chase VLP assay confirming that a decrease in POSH function leads to a complete or near-complete inhibition of VLP production. Accordingly, systems disclosed herein are effective for rapidly generating drug targets.

Detailed protocols for performing VLP assays and siRNA knockdown experiments are as follows.

Steady-State VLP Assay:

1. Objective:

Use RNAi to inhibit POSH gene expression and compare the efficiency of viral budding and GAG expression and processing in treated and untreated cells.

2. Study Plan:

HeLa SS-6 cells are transfected with mRNA-specific RNAi in order to knockdown the target proteins. Since maximal reduction of target protein by RNAi is achieved after 48 hours, cells are transfected twice - first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA.

3. Methods, Materials, Solutions

a. Methods

i. Transfections according to manufacturer's protocol and as described in procedure.

ii. Protein determined by Bradford assay.

iii. SDS-PAGE in Hoeffer miniVE electrophoresis system. Transfer in Bio-Rad mini-protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life Technologies | 11668-019 | 1112496 |
| (LF2000) | | | |
| OptiMEM | Life Technologies | 31985-047 | 3063119 |
| RNAi Lamin A/C | Self | 13 | |
| RNAi TSG101 688 | Self | 65 | |
| RNAi Posh 524 | Self | 81 | |
| plenv11 PTAP | Self | 148 | |
| plenv11 ATAP | Self | 149 | |
| Anti-p24 polyclonal | Seramun | | A-0236/5- |
| antibody | | | 10-01 |
| Anti-Rabbit Cy5 | Jackson | 144-175-115 | 48715 |
| conjugated antibody | | | |
| 10% acrylamide Tris- | Life Technologies | NP0321 | 1081371 |
| Glycine SDS-PAGE gel | | | |
| Nitrocellulose | Schleicher & | 401353 | BA-83 |
| membrane | Schuell | | |
| NuPAGE 20X transfer | Life Technologies | NP0006-1 | 224365 |
| buffer | | | |
| 0.45 μm filter | Schleicher & | 10462100 | CS1018-1 |
| | Schuell | | |

c. Solutions

| | Compound | Concentration |
|---|---|---|
| Lysis Buffer | Tris-HCl pH 7.6 | 50 mM |
| | MgCl₂ | 15 mM |
| | NaCl | 150 mM |
| | Glycerol | 10% |
| | EDTA | 1 mM |
| | EGTA | 1 mM |
| | ASB-14 (add immediately | 1% |
| | before use) | |
| 6X Sample | Tris-HCl, pH = 6.8 | 1 M |
| Buffer | Glycerol | 30% |
| | SDS | 10% |
| | DTT | 9.3% |
| | Bromophenol Blue | 0.012% |
| TBS-T | Tris pH = 7.6 | 20 mM |
| | NaCl | 137 mM |
| | Tween-20 | 0.1% |

4. Procedure

a. Schedule

Day

| 1 | 2 | 3 | 4 | 5 |
|---|---|---|---|---|
| Plate | Transfection I | Passage | Transfection II | Extract RNA |
| cells | (RNAi only) | cells | (RNAi and pNlenv) | for RT-PCR |
| | | (1:3) | (12:00, PM) | (post |
| | | | | transfection) |
| | | | Extract RNA for | Harvest VLPs |
| | | | RT-PCR | and cells |
| | | | (pre-transfection) | |

b. Day 1

Plate HeLa SS-6 cells in 6-well plates (35 mm wells) at concentration of 5 X105 cells/well.

c. Day2

2 hours before transfection replace growth medium with 2 ml growth medium without antibiotics.

Transfection I:

| | | | | | RNAi | A | B |
|---|---|---|---|---|---|---|---|
| | | | | | [20 μM] | OPtiMEM | LF2000 mix |
| Reaction | RNAi name | TAGDA # | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | Posh 524 | 81 | 2 | 50 | 12.5 | 500 | 500 |

Transfections:

Prepare LF2000 mix: 250 μl OptiMEM+5 μl LF2000 for each reaction. Mix by inversion, 5 times. Incubate 5 minutes at room temperature.

Prepare RNA dilution in OptiMEM (Table 1, column A). Add LF2000 mix dropwise to diluted RNA (Table 1, column B). Mix by gentle vortex. Incubate at room temperature 25 minutes, covered with aluminum foil.

Add 500 μl transfection mixture to cells dropwise and mix by rocking side to side.

Incubate overnight.

d. Day3

Split 1:3 after 24 hours. (Plate 4 wells for each reaction, except reaction 2 which is plated into 3 wells.)

e. Day4

2 hours pre-transfection replace medium with DMEM growth medium without antibiotics.

Transfection II

| | | | | A | B | | |
|---|---|---|---|---|---|---|---|
| | | | | Plasmid | RNAi | | |
| | | | | for 2.4 | [20 μM] | C | D |
| | | Plasmid | Plasmid | μg | for 10 nM | OPtiMEM | LF2000 mix |
| RNAi name | TAGDA # | Reactions | (μg/μl) | (μl) | (μl) | (μl) | (μl) |
| Lamin A/C | 13 | PTAP | 3 | 3.4 | 3.75 | 750 | 750 |
| Lamin A/C | 13 | ATAP | 3 | 2.5 | 3.75 | 750 | 750 |
| TSG101 688 | 65 | PTAP | 3 | 3.4 | 3.75 | 750 | 750 |
| Posh 524 | 81 | PTAP | 3 | 3.4 | 3.75 | 750 | 750 |

Prepare LF2000 mix: 250 μl OptiMEM+5 μl LF2000 for each reaction. Mix by inversion, 5 times. Incubate 5 minutes at room temperature.

Prepare RNA+DNA diluted in OptiMEM (Transfection II, A+B+C)

Add LF2000 mix (Transfection II, D) to diluted RNA+DNA dropwise, mix by gentle vortex, and incubate 1 h while protected from light with aluminum foil.

Add LF2000 and DNA+RNA to cells, 500 μl/well, mix by gentle rocking and incubate overnight.

f. Day 5

Collect samples for VLP assay (approximately 24 hours post-transfection) by the following procedure (cells from one well from each sample is taken for RNA assay, by RT-PCR).

g. Cell Extracts

i. Pellet floating cells by centrifugation (5min, 3000 rpm at 40° C.), save supernatant (continue with supernatant immediately to step h), scrape remaining cells in the medium which remains in the well, add to the corresponding floating cell pellet and centrifuge for 5 minutes, 1800 rpm at 40° C.

ii. Wash cell pellet twice with ice-cold PBS.

iii. Resuspend cell pellet in 100 μl lysis buffer and incubate 20 minutes on ice.

iv. Centrifuge at 14,000 rpm for 15 min. Transfer supernatant to a clean tube. This is the cell extract.

v. Prepare 10 μl of cell extract samples for SDS-PAGE by adding SDS-PAGE sample buffer to 1X, and boiling for 10 minutes. Remove an aliquot of the remaining sample for protein determination to verify total initial starting material. Save remaining cell extract at −80° C.

h. Purification of VLPs from cell media

i. Filter the supernatant from step g through a 0.45 m filter.

ii. Centrifuge supernatant at 14,000 rpm at 40 C for at least 2 h.

iii. Aspirate supernatant carefully.

iv. Re-suspend VLP pellet in hot (100° C. warmed for 10 min at least) 1X sample buffer.

v. Boil samples for 10 minutes, 100° C.

i. Western Blot analysis

i. Run all samples from stages A and B on Tris-Glycine SDS-PAGE 10% (120 V for 1.5 h.).

ii. Transfer samples to nitrocellulose membrane (65 V for 1.5 h.).

iii. Stain membrane with ponceau S solution.

iv. Block with 10% low fat milk in TBS-T for 1 h.

v. Incubate with anti p24 rabbit 1:500 in TBS-T o/n.

vi. Wash 3 times with TBS-T for 7 min each wash.

vii. Incubate with secondary antibody anti rabbit cy5 1:500 for 30 min.

viii. Wash five times for 10 min in TBS-T

ix. View in Typhoon gel imaging system (Molecular Dynamics/APBiotech) for fluorescence signal.

Exemplary RT-PCR Primers for POSH

Exemplary RT-PCR primers for POSH

| | | |
|---|---|---|
| Name | Position | Sequence |

| | | | | |
|---|---|---|---|---|
| Sense primer | POSH = 271 | 271 | 5′ CTTGCCTTGCCAGCATAC 3′ | (SEQ ID NO: 12) |
| Anti-sense primer | POSH = 926c | 926C | 5′ CTGCCAGCATTCCTTCAG 3′ | (SEQ ID NO: 13) |

siRNA duplexes:

| | | |
|---|---|---|
| siRNA No: | 153 | |
| siRNA Name: | POSH-230 | |
| Position in mRNA | 426-446 | |
| Target sequence: | 5′ AACAGAGGCCTTGGAAACCTG 3′ | SEQ ID NO: 14 |
| siRNA sense strand: | 5′ dTdTCAGAGGCCUUGGAAACCUG 3′ | SEQ ID NO: 15 |
| siRNA anti-sense strand: | 5′ dTdTCAGGUUUCCAAGGCCUCUG 3′ | SEQ ID NO: 16 |
| siRNA No: | 155 | |
| siRNA Name: | POSH-442 | |
| Position in mRNA | 638-658 | |
| Target sequence: | 5′ AAAGAGCCTGGAGACCTTAAA 3′ | SEQ ID NO: 17 |
| siRNA sense strand: | 5′ ddTdTAGAGCCUGGAGACCUUAAA 3′ | SEQ ID NO: 18 |
| siRNA anti-sense strand: | 5′ ddTdTUUUAAGGUCUCCAGGCUCU 3′ | SEQ ID NO: 19 |
| siRNA No: | 157 | |
| siRNA Name: | POSH-U111 | |
| Position in mRNA | 2973-2993 | |
| Target sequence: | 5′ AAGGATTGGTATGTGACTCTG 3′ | SEQ ID NO: 20 |
| siRNA snese strand: | 5′ dTdTGGAUUGGUAUGUGACUCUG 3′ | SEQ ID NO: 21 |
| siRNA anti-sense strand: | 5′ dTdTCAGAGUCACAUACCAAUCC 3′ | SEQ ID NO: 22 |
| siRNA No: | 159 | |
| siRNA Name: | POSH-U410 | |
| Position in mRNA | 3272-3292 | |
| Target sequence: | 5′ AAGCTGGATTATCTCCTGTTG 3′ | SEQ ID NO: 23 |
| siRNA sense strand: | 5′ ddTdTGCUGGAUUAUCUCCUGUUG 3′ | SEQ ID NO: 24 |
| siRNA anti-sense strand: | 5′ ddTdTCAACAGGAGAUAAUCCAGC 3′ | SEQ ID NO: 25 |

Protocol For Assessing POSH siRNA Effects on the Kinetics of VLP Release

A1. Transfections

1. One day before transfection plate cells at a concentration of 5×10⁶ cell/well in 15 cm plates.

2. Two hours before transfection, replace cell media to 20 ml complete DMEM without antibiotics.

3. DNA dilution: for each transfection dilute 62.5 μl RNAi in 2.5 ml OptiMEM according to the table below. RNAi stock is 20 μM (recommended concentration: 50 nM, dilution in total medium amount 1:400).

4. LF 2000 dilution: for each transfection dilute 50 μl lipofectamine 2000 reagent in 2.5 ml OptiMEM.

5. Incubate diluted RNAi and LF 2000 for 5 minutes at RT.

6. Mix the diluted RNAi with diluted LF2000 and incubated for 20-25 minutes at RT.

7. Add the mixure to the cells (drop wise) and incubate for 24 hours at 37° C. in CO₂ incubator.

8. One day after RNAi transfection split cells (in complete MEM medium to 2 15 cm plate and 1 well in a 6 wells plate)

9. One day after cells split perform HIV transfection according to SP 30-012-01.

10. 6 hours after HIV transfection replace medium to complete MEM medium.

Perform RT-PCR for POSH to assess degree of knockdown.

A2. Total RNA purification.

1. One day after transfection, wash cells twice with sterile PBS.

2. Scrape cells in 2.3 ml/200 μl (for 15 cm plate/1 well of a 6 wells plate) Tri reagent (with sterile scrapers) and freeze in −70° C.

| | Chase time | | |
|---|---|---|---|
| Treatment | (hours) | Fraction | Labeling |
| Control = WT | 1 | Cells | A1 |
| | | VLP | A1 V |
| | 2 | Cells | A2 |
| | | VLP | A2 V |
| | 3 | Cells | A3 |
| | | VLP | A3 V |
| | 4 | Cells | A4 |
| | | VLP | A4 V |
| | 5 | Cells | A5 |
| | | VLP | A5 V |
| Posh + WT | 1 | Cells | B1 |
| | | VLP | B1 V |
| | 2 | Cells | B2 |
| | | VLP | B2 V |
| | 3 | Cells | B3 |
| | | VLP | B3 V |
| | 4 | Cells | B4 |
| | | VLP | B4 V |
| | 5 | Cells | B5 |
| | | VLP | B5 V |

B. Labeling

1. Take out starvation medium, thaw and place at 37° C.

2. Scrape cells in growth medium and transfer gently into 15 ml conical tube.

3. Centrifuge to pellet cells at 1800 rpm for 5 minutes at room temperature.

4. Aspirate supernatant and let tube stand for 10 sec. Remove the rest of the supernatant with a 200 μl pipetman.

5. Gently add 10 ml warm starvation medium and resuspend carefully with a 10 ml pipette, up and down, just turning may not resolve the cell pellet).

6. Transfer cells to 10 cm tube and place in the incubator for 60 minutes. Set an Eppendorf thermo mixer to 37° C.

7. Centrifuge to pellet cells at 1800 rpm for 5 minutes at room temperature.

8. Aspirate supernatant and let tube stand for 10 sec. Remove the rest of the supernatant with a 200 μl pipetman.

9. Cut a 200 μl tip from the end and resuspend cells (˜1.5 10⁷ cells in 150 μl RPIM without Met, but try not to go over 250 μl if you have more cells) gently in 150 μl starvation medium. Transfer cells to an Eppendorf tube and place in the thermo mixer. Wait 10 sec and transfer the rest of the cells from the 10 ml tube to the Eppendorf tube, if necessary add another 50 μl to splash the rest of the cells out (all specimens should have the same volume of labeling reaction!).

10. Pulse: Add 50 μl of ³⁵S-methionine (specific activity 14.2 μCi/μl), tightly cup tubes and place in thermo mixer. Set the mixing speed to the lowest possible (700 rpm) and incubate for 25 minutes.

11. Stop the pulse by adding 1 ml ice-cold chase/stop medium. Shake tube very gently three times and pellet cells at 6000 rpm for 6 sec.

12. Remove supernatant with a 1 ml tip. Add gently 1 ml ice-cold chase/stop medium to the pelleted cells and invert gently to resuspend.

13. Chase: Transfer all tubes to the thermo mixer and incubate for the required chase time (830:1,2,3,4 and 5 hours; 828: 3 hours only). At the end of total chase time, place tubes on ice, add 1 ml ice-cold chase/stop and pellet cells for 1 minute at 14,000 rpm. Remove supernatant and transfer supernatant to a second eppendorf tube. The cell pellet freeze at −80° C., until all tubes are ready.

14. Centrifuge supernatants for 2 hours at 14,000 rpm, 4° C. Remove the supernatant very gently, leave 20 μl in the tube (labeled as V) and freeze at −80° C. until the end of the time course.

All steps are done on ice with ice-cold buffers

15. When the time course is over, remove all tubes form −80° C. Lyse VLP pellet (from step 14) and cell pellet (step 13) by adding 500 μl of lysis buffer (see solutions), resuspend well by pipeting up and down three times. Incubate on ice for 15 minutes, and spin in an eppendorf centrifuge for 15 minutes at 4° C., 14,000 rpm. Remove supernatant to a fresh tube, discard pellet.

16. Perform IP with anti-p24 sheep for all samples.

C. Immunoprecipitation

1. Preclearing: add to all samples 15 μl ImnunoPure PlusG (Pierce). Rotate for 1 hour at 4° C. in a cycler, spin 5 min at 4° C., and transfer to a new tube for IP.

2. Add to all samples 20 μl of p24-protein G conjugated beads and incubate 4 hours in a cycler at 4° C.

3. Post immunoprecipitations, transfer all immunoprecipitations to a fresh tube.

4. Wash beads once with high salt buffer, once with medium salt buffer and once with low salt buffer. After each spin don't remove all solution, but leave 50 μl solution on the beads. After the last spin remove supernatant carefully with a loading tip and leave ˜10 μl solution.

5. Add to each tube 20 μI 2× SDS sample buffer. Heat to 70° C. for 10 minutes.

6. Samples were separated on 10% SDS-PAGE.

7. Fix gel in 25% ethanol and 10% acetic acid for 15 minutes.

8. Pour off the fixation solution and soak gels in Amplify solution (NAMP 100 Amersham) for 15 minutes.

9. Dry gels on warm plate (60-80° C.) under vacuum.

10. Expose gels to screen for 2 hours and scan.

### Example 5

Identification of Drug Targets For Anti-Neoplastic Agents

A database of greater than 500 E3 proteins is assembled. The database contains many of the proteins presented in Table 2. A subset of proteins is selected based on various characteristics, such as the presence of certain domains. The expression of genes encoding the proteins is assessed in cancerous and non-cancerous tissues to identify genes of the database that are overexpressed or underexpressed in cancerous tissues. Examples of cancerous and non-cancerous tissues to be tested include: lung, laryngopharynx, pancreas, liver, rectum, colon, stomach, breast, cervix, uterus, ovary, testes, prostate and skin.

Genes that are identified as overexpressed in cancer are subjected to siRNA knockdown in a cancerous cell line, such as HeLa cells. If the knockdown decreases proliferation of the cancerous cell line, the gene and the encoded protein are targets for developing anti-neoplastic agents.

POSH is overexpressed in certain cancerous tissues, and POSH siRNA decreases proliferation of HeLa cells.

Incorporation By Reference

All of the patents and publications cited herein are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

We claim:

1. A method of identifying a potential drug target, comprising:
providing a database comprising nucleic acid or protein sequences, wherein said sequences are annotated with potential disease-associations of said sequences;
providing an assay for measuring the disease characteristic of a disease potentially associated to any one of said sequences;
decreasing expression or activity of at least one of the nucleic acid or protein sequences provided in the database; and
determining whether the decreased expression or activity results in a change in said assay wherein a change in said assay is indicative that said nucleic acid or protein sequence is a potential drug target for the associated disease.

2. A method of identifying a potential drug target comprising:
providing a database comprising nucleic acid or protein sequences, wherein said sequences are annotated with potential disease-associations of said sequences;
providing an assay for measuring the disease characteristic of a disease potentially associated to any one of said sequences;
increasing expression or activity of at least one of the nucleic acid or protein sequences provided in the database; and
determining whether the increased expression or activity results in a change in said assay wherein a change in said assay is indicative that said nucleic acid or protein sequence is a potential drug target for the associated disease.

3. A method of identifying a potential drug target comprising:
providing a database comprising nucleic acid or protein sequences, wherein said sequences are annotated with potential disease-associations of said sequences;
determining differential expression or activity of said nucleic acid or protein sequences in a cell exhibiting a disease characteristic of a potential associated disease and a corresponding normal cell;
decreasing expression or activity of said nucleic acid or protein sequences; and
determining the effect of decreased expression or activity on said cell exhibiting disease characteristics of the associated disease, wherein a change in said disease characteristics is indicative that said nucleic acid or protein sequence is a potential drug target for said associated disease.

4. A method of identifying a potential drug target comprising:
providing a database comprising nucleic acid or protein sequences, wherein said sequences are annotated with potential disease-associations of said sequences;
determining differential expression of said nucleic acid or protein sequences in a cell exhibiting disease characteristics of a potential associated disease and a corresponding normal cell;
increasing expression or activity of said nucleic acid or protein sequence; and
determining the effect of increased expression or activity on said cell exhibiting disease characteristics of the associated disease, wherein a change in said disease characteristics is indicative that said nucleic acid or protein sequence is a potential drug target for said associated disease.

5. The method of any one of claims 1-4, further comprising creating the database.

6. The method of any one of claims 1-4, wherein said database optionally contains domain analysis.

7. The method of claim 5, wherein creating the database comprises:
receiving a first set of information corresponding to a protein or nucleic acid;
receiving a second set of information identifying a characteristic of said nucleic acid or protein; and
conducting a clustering analysis to determine how said protein or nucleic acid should be clustered based on the first and second sets of information.

8. The method of claim 7, wherein the first set of information comprises sequence information and/or structural information.

9. The method of claim 7, wherein the second set of information comprises domain information.

10. The method of claim 9, wherein the second set of information indicates the presence or absence of one or more domains selected from the group of: Hect, Ring, Ubox, Fbox and PHD.

11. The method of any one of claims 1-4, wherein the nucleic acid or protein sequence is a human E3 sequence.

12. The method of any one of claims 1-4, wherein the potential disease associations are selected from the group consisting of viral diseases, proliferative disorders, and ubiquitin-mediated disorders.

13. The method of any one of claims 1-2, wherein the assay determines a disease characteristic of an associated disease.

14. The method of claim 13, wherein said disease characteristic is assessed by determining whether said protein interacts with an interacting-protein, and wherein said interacting-protein undergoes abnormal degradation in the disease characteristic.

15. The method of claim 13, wherein said disease characteristic is assessed by determining the cellular localization of said protein.

16. The method of claim 13, wherein said disease characteristic is assessed by determining the biological activity of said protein.

17. The method of claim 13, wherein the protein is a E3 protein.

18. The method of claim 17, wherein said disease characteristic is assessed by determining a biological activity of said E3 protein.

19. The method of claim 18, wherein the biological activity is the ligase activity of said E3 protein.

20. The method of claim 18, wherein said disease characteristic is assessed by determining whether said E3 interacts with a substrate that is ubiquitinated in the disease characteristic.

21. The method of claim 12, wherein said associated disease is a retroviral infection.

22. The method of claim 21, wherein said retroviral infection is HIV infection.

23. The method of claim 21, wherein said assay comprises determining the release of virus like particles (VLP) from infected cells.

24. The method of claim 23, wherein decreasing expression or activity of an E3 protein results in a change in the release of said VLPs.

25. The method of claim 24, wherein said E3 protein contains a WW domain.

26. The method of claim 24, wherein said E3 protein contains a HECT domain.

27. The method of claim 24, wherein said E3 protein contains a SH3 domain.

28. The method of claim 24, wherein said E3 protein contains a RING domain.

29. The method of any one of claims 1 or 3, wherein expression of said nucleic acid sequence is decreased using RNAi.

30. The method of any one of claims 1 or 3, wherein expression of said nucleic acid sequence is decreased using an antisense oligonucleotide construct.

31. The method of any one of claims 1 or 3, wherein expression of said nucleic acid sequence is decreased using ribozyme.

32. The method of any one of claims 1 or 3, wherein expression of said nucleic acid sequence is decreased using a DNA enzyme.

33. The method of claim 4, wherein the protein is a E3 protein.

34. The method of claim 33, wherein decreased expression of said E3 is indicative of a disease characteristic.

35. The method of claim 34, wherein said E3 is a tumor suppressor and the disease characteristic is tumorigenesis.

36. The method of claim 35, wherein an increase in expression or activity of said E3 protein results in a gain of function phenotype.

37. The method of claims 36, wherein said E3 is a potential drug target.

38. The method of claim 37, wherein the substrate of said E3 is also a potential drug target.

39. The method of claim 5, wherein access to the database is provided to subscribers.

40. A method for determining whether a test sequence is a potential drug target, comprising:
providing a database comprising nucleic acid or protein sequences, wherein said sequences are annotated with potential disease-associations of said sequences;
comparing said test sequence to the sequences provided in said database and predicting potential disease associations;
validating the predicted disease association by decreasing the activity of said nucleic acid or protein sequences; and
updating the database to include the test sequence and associated annotations.

41. A method of identifying a therapeutic ribozyme for treating viral infections comprising:
(a) providing an E3 drug target for treating viral infections;
(b) administering a ribozyme to decrease expression of said E3 in an infected cell;
(c) determining the release of virus like particles from said infected cell; and
wherein a decrease in the release of virus like particles is indicative that said ribozyme is a therapeutic ribozyme for treating said viral infections.

42. A method of identifying a therapeutic ribozyme for treating cancer comprising:
(a) providing an E3 drug target for treating cancer;
(b) administering a ribozyme to decrease expression of said E3 in a tumor cell;
(c) determining the rate of proliferation of said tumor cell;
wherein a decrease in the rate of proliferation is indicative that said ribozyme is a therapeutic ribozyme for treating said proliferative diseases.

43. A method of identifying a therapeutic RNAi construct for treating viral infections comprising:
(a) providing an E3 drug target for treating viral infections;
(b) administering a RNAi construct to decrease expression of said E3 in an infected cell;
(c) determining the release of virus like particles from said infected cell; and
wherein a decrease in the release of virus like particles is indicative that said RNAi construct is a therapeutic RNAi construct for treating said viral infections.

42. A method of identifying a therapeutic RNAi construct for treating cancer comprising:
(a) providing an E3 drug target for treating cancer;
(b) administering a RNAi construct to decrease expression of said E3 in a tumor cell;
(c) determining the rate of proliferation of said tumor cell;
wherein a decrease in the rate of proliferation is indicative that said RNAi construct is a therapeutic ribozyme for treating said proliferative diseases.

43. A method of screening E3 proteins as potential drug targets, comprising:
selecting an E3 protein;
decreasing expression or activity of said E3 protein in an viral-infected cell;
determining the release of virus like particles upon decreasing the expression or activity of said E3;
wherein a decrease the release of the virus like particles is indicative that said E3 protein is a potential drug target.

44. A method of creating a database of E3 proteins or nucleic acids, comprising:
receiving a first set of information corresponding to a protein or nucleic acid;
receiving a second set of information identifying a characteristic of said nucleic acid or protein sequence; and
conducting a clustering analysis to determine how said protein or nucleic acid sequences should be clustered based on the first and second sets of information.

45. The method of claim 44, wherein the first set of information comprises sequence information and/or structural information.

46. The method of claim 44, wherein the second set of information comprises domain information.

47. The method of claim 44, wherein the second set of information indicates the presence or absence of one or more domains selected from the group of: Hect, Ring, Ubox, Fbox and PHD.

48. The method of claim 47, wherein all protein and nucleic acid sequences comprising one or more domains selected from the group of: Hect, Ring, Ubox, Fbox and PHD are included within said database.

49. The method of claim 48, wherein the protein and nucleic acid sequences are further clustered based on the presence or absence of said domains.

50. The method of claim 48, wherein the protein and nucleic acid sequences are further clustered based on certain disease associations.

51. The method of claim 48, wherein the protein and nucleic acid sequences are further clustered based on the presence or absence of interacting motifs.

52. The method of claim 48, wherein the protein and nucleic acid sequences are further clustered based on one or more of the following: homology modeling, secondary structure, threading, transmembrane helices, signal peptide domains, and protein localization signals.

53. The method of claim 48, wherein said E3 sequences are evaluated as potential drug targets.

54. The method of claim 48, wherein said E3 sequences are screened is biological assays for testing disease associations.

55. A method of creating a database of proteins or nucleic acid sequences containing the RING domain, comprising:
receiving a first set of information corresponding to a protein or nucleic acid;
receiving a second set of information identifying a characteristic of said nucleic acid or protein sequence; and
conducting a clustering analysis to determine how said protein or nucleic acid sequences should be clustered based on the first and second sets of information.

56. The method of claim 55, wherein all protein and nucleic acid sequences comprising one or more Ring domains included within said database.

57. A method of screening an E3 protein as potential drug target, comprising:
selecting an E3 protein;
decreasing expression or activity of said E3 protein in a tumor cell;
determining the rate of proliferation of said tumor cell upon decreasing the expression or activity of said E3;
wherein a decrease in the rate of proliferation is indicative that said E3 protein is a potential drug target.

58. A method of screening an E3 protein as a potential drug targets, comprising:
selecting an E3 protein;
decreasing expression or activity of said E3 protein in a diseased cell;
determining the effect of decreasing the expression or activity of said E3 on a Ubiquitin-mediated disorder;
wherein a change is indicative that said E3 protein is a potential drug target.

59. The method of any one of claims 1 or 3, wherein expression or activity is decreased by using a dominant negative mutant.

60. The method of any one of claims 1 or 3, wherein expression or activity is decreased by using a small molecule.

61. A method of identifying a potential drug target for an associated disease comprising:
(a) conducting a structure-function analysis to determine domain information and/or structural information involved in disease associations;
(b) providing a database comprising nucleic acid or protein sequence;
(c) selecting sequences containing the domains and/or structural information relevant to disease associations;
(d) providing an assay for measuring the disease characteristic;
(e) decreasing the expression or activity of the nucleic acid or protein sequence selected in step (c); and
(f) determining whether the decreased expression or activity results in change in said assay;
wherein a change in the disease characteristic is indicative of a potential drug target.

62. A method of identifying a potential drug target for an associated disease comprising:
(a) conducting a structure-function analysis to determine domain information and/or structural information involved in disease associations;
(b) providing a database comprising nucleic acid or protein sequence;
(c) selecting sequences containing the domains and/or structural information relevant to disease associations;
(d) providing an assay for measuring the disease characteristic;
(e) increasing the expression or activity of the nucleic acid or protein sequence selected in step (c); and
(f) determining whether the increased expression or activity results in change in said assay;
wherein a change in the disease characteristic is indicative of a potential drug target.

63. The method of claim 61 or claim 62, wherein the protein and nucleic acid sequences are E3 sequences.

64. The method of claim 63, wherein the protein and nucleic acid sequences comprise one or more domains selected from the group of: Hect, Ring, Ubox, Fbox and PHD.

65. The method of claim 64, wherein the disease associations are selected from the group consisting of viral diseases, proliferative disorders, and ubiquitin-mediated disorders.

66. The method of claim 65, wherein the assay determines a disease characteristic of an associated disease.

67. The method of claim 66, wherein said disease characteristic is assessed by determining whether said protein interacts with an interacting-protein, and wherein said interacting-protein undergoes abnormal degradation in the disease characteristic.

68. The method of claim 66, wherein said disease characteristic is assessed by determining the cellular localization of said protein.

69. The method of claim 66, wherein said disease characteristic is assessed by determining whether said E3 interacts with a substrate that is ubiquitinated in the disease characteristic.

70. The method of claim 61, wherein expression of said nucleic acid sequence is decreased using RNAi construct.

71. The method of claim 61, wherein expression of said nucleic acid sequence is decreased using an antisense oligonucleotide construct.

72. The method of claim 61, wherein expression of said nucleic acid sequence is decreased using ribozyme.

73. The method of claim 61, wherein expression of said nucleic acid sequence is decreased using a DNA enzyme.

74. The method of claim 61, wherein activity of said protein is decreased by using a dominant negative mutant.

75. The method of claim 61, wherein expression or activity is decreased by using a small molecule.

76. The method of any one of claims 5, 44, or 55, wherein said database comprises at least 20, 25, 50, 75, 100, 125, 150, 200, 250, or 300 sequences.
